(19) United States
(12) Patent Application Publication
(10) Pub. No.: US 2025/0270492 A1
(43) Pub. Date: Aug. 28, 2025

(54) SYSTEM FOR PRODUCING EXTRACELLULAR VESICLES AND METHOD FOR PRODUCING EXTRACELLULAR VESICLES

(71) Applicant: AMOGREENTECH CO., LTD., Gimpo-si (KR)

(72) Inventors: Hee Sung PARK, Gimpo-si (KR); Su-Yeon LEE, Gimpo-si (KR); In-Yong SEO, Gimpo-si (KR); Ji Young KIM, Gimpo-si (KR)

(73) Assignee: AMOGREENTECH CO., LTD., Gimpo-si (KR)

(21) Appl. No.: 18/858,354

(22) PCT Filed: Apr. 20, 2023
(86) PCT No.: PCT/KR2023/005395

§ 371 Date: Oct. 24, 2024

(30) Foreign Application Priority Data
Apr. 20, 2022 (KR) 10-2022-0048714

(51) Int. Cl. C12M 1/26; C12M 1/00; C12M 1/12; C12M 1/33; C12M 1/34

(52) U.S. Cl. CPC C12M 33/14; C12M 25/06; C12M 29/00; C12M 41/34; C12M 45/02; C12M 47/04

(57) ABSTRACT

A system for producing extracellular vesicles is provided. The system includes: a circulating culture medium type extracellular vesicle production unit that cultures cells with a circulating medium to produce a culture containing extracellular vesicles; and a separation unit that separates and obtains extracellular vesicles having a size of 200 nm or less, including exosomes, from any one or more stock solutions of the harvested culture and a cell disruption solution derived from the cultured cells. According to this, the production of target extracellular vesicles can be increased relative to the number of cells to be cultured, and the loss of target extracellular vesicles can be reduced from the produced culture and cultured cells, such that the target extracellular vesicles can be separated and obtained in excellent yield.

### CROSS REFERENCE TO THE RELATED APPLICATIONS

This application is the national phase entry of International Application No. PCT/KR2023/005395, filed on Apr. 20, 2023, which is based upon and claims priority to Korean Patent Application No. 10-2022-0048714, filed on Apr. 20, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBLO069_Sequence_Listing.xml, created on Oct. 18, 2024, and is 4,109 bytes in size.

### TECHNICAL FIELD

The present invention relates to a system for producing extracellular vesicles and a method for producing extracellular vesicles.

### BACKGROUND

All cells are known to exchange information with other cells or the external environment through various materials secreted from the external environment, for example, soluble factors such as cytokines, chemokines, hormones, and neurotransmitters.

Recently, information exchange through extracellular vesicles (EVs) is attracting attention in relation to information exchange between cells or between cells and the extracellular environment. EVs are nano-sized vesicles surrounded by a lipid bilayer that are secreted out of cells.

The EVs are called various names such as exosomes, microvesicles, ectosomes, microparticles, membrane vesicles, nanovesicles, and outer membrane vesicles according to their origin, secretion mechanisms, and sizes. For example, mammalian EVs are classified into exosomes that are produced when the endosomal membrane is pulled inward to create intraluminal vesicles during the maturation of multivesicular endosomes (MVEs), and then the MVEs fuse with the cell surface and the intraluminal vesicles are secreted out of the cell, and microvesicles that are secreted out of the cell when the plasma membrane buds out and is separated.

EVs include various materials with biological activity, such as proteins, lipids, nucleic acids, and metabolites, reflecting the state of the cells from which they originate. In addition, EVs are reported to be present in cell culture fluid and various body fluids such as blood, urine, saliva, tears, semen, breast milk, and ascites, and perform various physiological/pathological functions, and research is actively being conducted to approach them as a treatment for various diseases.

However, since eukaryotic cells secrete only 100 to 1000 exosomes per cell, the low production rate of EVs including exosomes is a problem.

In addition, the amount of target lost in the separation of targets, such as EVs including exosomes, from a culture containing EVs including exosomes is large, resulting in a poor separation yield.

As a result, there is a need for a method for mass production and separation of target EVs from cells.

### SUMMARY

### Technical Problem

In view of the above, the present invention is directed to providing an EV production system and an EV production method, which can produce a large amount of EVs relative to the number of cultured cells, and separate target EVs therefrom.

The present invention is also directed to providing an EV production system and an EV production method, which can reduce the loss of target EVs and improve the separation yield of EVs during the separation of the target EVs from the obtained culture.

### Technical Solution

In one aspect, the present invention provides an EV production system, which includes a medium circulation-type extracellular vesicle production unit which cultures cells in a circulating medium and produces a culture containing EVs; and a separation unit which separates and obtains EVs with a size of 200 nm or less including exosomes from one or more stock solutions selected from the recovered culture and a cell lysate derived from the cultured cells.

According to one embodiment of the present invention, the medium circulation-type EV production unit may include a cell culture unit, a medium storage unit, a circulation pump provided so that a medium stored in the medium storage unit circulates between the cell culture unit and the medium storage unit, and a gas supply unit supplying a gas to the medium provided to the cell culture unit.

In addition, the cell culture unit may include a culture housing with a culture space, and a plurality of plate-shaped cell culture supports spaced 5.0 mm or less apart in multiple stages inside the culture housing.

In addition, the cell culture support may include a fiber web and a bioactive peptide fixed on the surface of the fiber web.

In addition, the separation unit may include a first filter unit for separating and obtaining first particles with a size of 200 nm or less from a stock solution, a second filter unit for separating and removing second particles with a size of less than 50 nm from the separated first particles, and a recovery unit for storing separated EVs with a size of 50 to 200 nm.

In addition, the separation unit may further include a third filter unit for separating third particles with a size exceeding 200 nm between the second filter unit and the recovery unit.

In addition, the second filter unit may be configured to employ one or more of tangential flow filtration and size exclusion chromatography.

In addition, the separation unit may further include a cell lysis unit for lysing cells by applying a physical force to the cultured cells or a culture containing the cultured cells, which are supplied from the medium circulation-type EV production unit, and the cell lysate produced by the cell lysis unit may be supplied to the first filter unit.

In addition, the cell lysis unit may include an inlet through which a lysis target is supplied, an outlet through which the cell lysate is discharged, at least three fluid channels through which the lysis target passes, which connect the inlet and the outlet, and have a width of 1 mm or less and a plurality of partitions disposed to block each fluid channel and having a plurality of microchannels passing through the partitions to lyse cells in the lysis target. The plurality of partitions include a first partition and a second partition disposed closer to the outlet than the first partition, and the width of each microchannel formed in the first partition may be 10 to 20 μm, and the width of each microchannel formed in the second partition may be 1 to 5 μm.

In addition, the present invention provides an EV production method, which includes (1) culturing cells in a circulating medium and producing a culture containing EVs; and (2) separating and obtaining EVs with a size of 200 nm or less, which include exosomes, from a stock solution including any one or more selected from the culture and a cell lysate derived from the cultured cells.

According to one embodiment of the present invention, Step (1) may include a cell seeding step for supplying cells mixed in a medium to a cell culture unit, a cell culture step for producing EVs by circulating the medium so that the medium discharged through one side of the cell culture unit is supplied back to the cell culture unit, and a step of obtaining a culture containing the produced EVs.

In addition, the medium may be circulated at a rate of 20 to 100 mL/min, maintained at pH 7.5 to 8.0 with a CO₂ concentration of 4.5 to 6%, and the cells may be cultured at 37 to 38° C.

In addition, the cells may include one or more types of cells selected from the group consisting of immune cells, embryonic stem cells, adult stem cells, induced pluripotent step cells (iPS cells), and progenitor cells.

In addition, no bubbles may be contained in the medium supplied to the cell culture unit.

In addition, the cell culture unit may include a plurality of plate-shaped cell culture supports whose main surfaces face other at a predetermined interval, and the cell seeding step may include orienting the cell culture unit so that the main surface of the plurality of plate-shaped cell culture supports are substantially parallel to the ground to settle the cells onto the cell culture supports.

In addition, the cell culture step may include culturing cells by orienting the cell culture unit so that the main surfaces of the plurality of plate-shaped cell culture supports are substantially perpendicular to the ground.

In addition, the cells may be cultured on the plurality of plate-shaped cell culture supports whose main surfaces face other at a predetermined interval and the main surfaces are arranged perpendicular to the ground, and the medium may be circulated to flow through the space between the cell culture supports in a bottom-to-top direction from the ground.

In addition, in the cell culture step, after the cells are proliferated to occupy 80 to 90% of the total effective cell culture area in the cell culture unit, a medium that does not contain foreign EVs may be circulated.

In addition, Step (2) may include a first filtration step for separating a first filtrate that includes first particles with a size of 200 nm or less, including EVs, from the stock solution; and a second filtration step for separating and removing second particles with a size of less than 50 nm from the first filtrate.

In addition, the second filtration step may be performed on the first filtrate diluted 3.0-fold to 15.0-fold with a buffer.

In addition, the first filtration step may be performed by multi-stage filtration of the stock solution to produce a filtrate containing smaller particles at each stage, or performed on the stock solution diluted 3.0-fold to 15.0-fold with a buffer.

In addition, a third filtration step may be further performed to separate and remove third particles with a size exceeding 200 nm from the second filtrate that has undergone the second filtration step.

In addition, the present invention provides a medium composition for cell culture, which includes particles with a size of 200 nm or less, including EVs, as a cell culture additive.

Hereinafter, terms used herein will be described.

The term “extracellular vesicle” used herein includes all materials collectively referred to as a cell membrane-derived endoplasmic reticulum, an ectosome, a shedding vesicle, a microparticle, an exosome, a microvesicle, or an outer membrane vesicle, and refers to a particle-shaped structure in which various biomolecules, such as proteins with various functions secreted (released) (e.g., various types of growth factors, chemokines, cytokines, transcription factors, etc.), RNAs (mRNA, miRNA, etc.), and lipids, are encapsulated in the cell membrane of a lipid bilayer identical to the cell membrane of the cells from which they are derived.

In addition, the term “stem cell” used herein is an undifferentiated cell at a stage before differentiation into each cell that constitutes a tissue, and a generic term for cells with the ability to differentiate into specific cells under specific differentiation stimuli (environment).

### Advantageous Effects

According to the present invention, the production amount of target EVs can increase relative to the number of cultured cells. In addition, it is possible to separate and obtain target EVs with excellent yield by reducing the loss of produced EVs from the culture. Furthermore, since the production and separation of target EVs can be performed continuously, an automated process for mass-producing, separating and obtaining target EVs is possible.

### BRIEF DESCRIPTION OF THE DRAWINGS

FIG. 1 is a schematic diagram showing an extracellular vesicle (EV) production unit according to one embodiment of the present invention,

FIG. 2 is a diagram showing a cell culture unit that can be applied to the EV production unit according to one embodiment of the present invention,

FIG. 3 is an exploded diagram of FIG. 2,

FIG. 4 is a cross-sectional view in the A-A direction of FIG. 2,

FIG. 5 is an exploded view of the assembly of the cell culture supports of FIG. 3,

FIGS. 6A-6B show a cross-sectional view of cell culture supports that can be applied to a cell culture unit according to one embodiment of the present invention,

FIG. 7 is a schematic diagram showing a separation unit according to one embodiment of the present invention,

FIG. 8 is a conceptual diagram illustrating a tangential flow filtration method that can be applied to a separation unit according to one embodiment of the present invention,

FIGS. 9 and 10 are schematic diagrams showing separation units according to various embodiments of the present invention,

FIG. 11 is a conceptual diagram illustrating the separation method of size exclusion chromatography that can be applied to one embodiment of the present invention,

FIG. 12 is a schematic diagram illustrating the circulation flow of a medium circulating inside a cell culture unit in the process of producing a culture containing EVs according to one embodiment of the present invention, and

FIG. 13 is a plan view of a cell lysis unit according to one embodiment of the present invention.

### DETAILED DESCRIPTION OF THE EMBODIMENTS

Hereinafter, exemplary embodiments of the present invention will be described in detail with reference to the accompanying drawings so that those of ordinary skill in the art to which the present invention belongs can easily implement them. The present invention may be implemented in various different forms and is not limited to the embodiments described herein. To clearly explain the present invention in the drawings, parts that are not related to the description are omitted, and the like reference numerals are added to the same or similar components throughout the specification.

The EV production method according to one embodiment of the present invention may include (1) a step of producing a culture containing EVs by culturing cells in a circulating medium and (2) a step of separating and obtaining EVs with a size of 200 nm or less, including exosomes, from a stock solution containing one or more of the culture and a cell lysate derived from the cultured cells.

First, in Step (1) of the present invention, a step of producing a culture containing EVs by culturing cells in a circulating medium is performed.

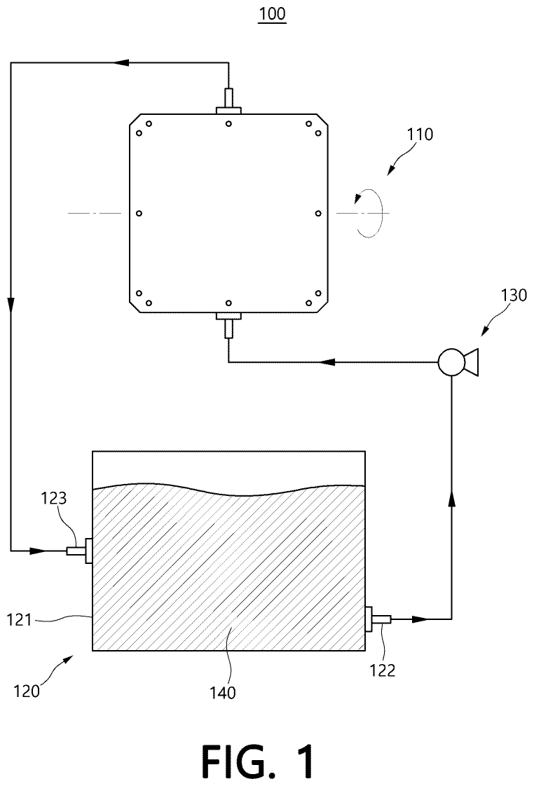

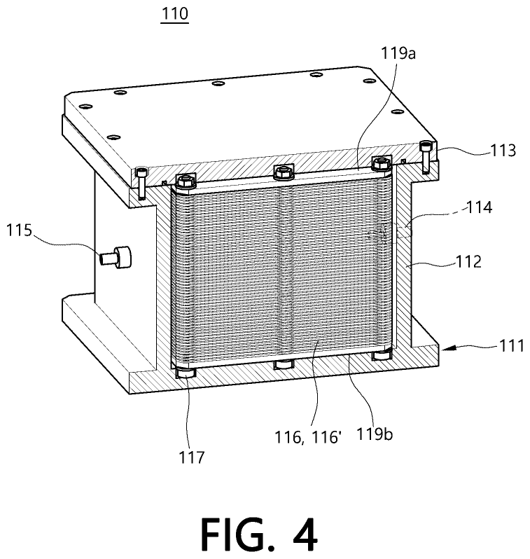

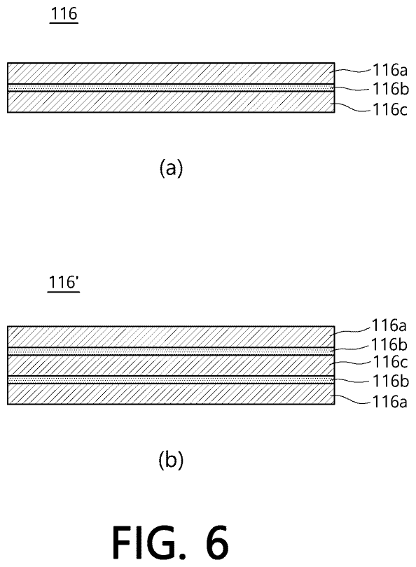

Step (1) may be performed using a device or system configured to continuously supply and discharge a medium in the space where cells are cultured. Preferably, Step (1) may be performed using a medium circulation-type EV production unit 100 including a cell culture unit 110, a medium storage unit 120, and a circulation pump 130, as shown in FIGS. 1 to 6B. In addition, the cell culture unit 110, the medium storage unit 120, and the circulation pump 130 may be disposed in an incubator with a predetermined internal space in which the temperature is constantly maintained, and the incubator may include an air conditioning system to maintain a constant temperature in the internal space.

The cell culture unit 110 may be a space where cells are seeded, cultured, and produce EVs, and include cell culture supports 116 and 116′ where the cells are attached and cultured. The cell culture supports 116 and 116′ may be any known plate-shaped cell culture support without limitation, and as an example, the cell culture supports may be integrated with a known polymer compound film, fiber web, or fiber web integrated with a film. Specifically, the cell culture supports may be a single or several sheets of fiber webs alone, a single or several films alone, or a laminate in which one or several fiber webs or one or several films are combined. Here, laminate-type cell culture supports 116 and 116′ may be combined via an adhesive 116B such as a silicone material or combined by partial melting of the film or fiber web without an adhesive when a nanofiber web 116A and a film 116C are combined.

The fiber web 116A may be formed of, for example, fibers with an average diameter of less than 1.5 μm, and as another example, formed of fibers with an average diameter of 10 nm to less than 1.5 μm, and may include a fiber web 116A having a basis weight of 1 to 20 g/m². The support fibers constituting the fiber web 116A may include any one or more of conventional materials used in cell culture, for example, polycarbonate (PC), polyacrylonitrile (PAN), polyurethane (PU), polylactic acid (PLA), poly(lactic-co-glycolic acid) (PLGA), polycaprolactone (PCL), polystyrene (PS), polyethersulfone (PES), and a fluorine-based compound (e.g., PVDF).

In addition, the film 116C may be implemented as a film by a known method. For example, the film-forming component may be melted and extruded through a die, or after melting the film-forming component, it may be dissolved and then formed on a substrate to a predetermined thickness through a conventional coating method. In addition, the film 116C may surface-modified by plasma treatment to improve hydrophilicity, and any known method can be used as the surface modification method for improving hydrophilicity. In addition, after cells are loaded, microcurves or grooves may be formed on the surface to have a certain surface roughness to improve the adhesion between the film surface and the cells. In addition, the film 116C may be polyester, for example, polyethylene terephthalate, polycarbonate, or polyurethane.

In addition, the cell culture supports 116 and 116′ have a thickness of preferably 200 to 800 μm, more preferably, 250 to 700 μm, and even more preferably, 300 to 520 μm. When the thickness is less than 200 μm, shaking and shape deformation may occur due to the medium flow passing through the space between the cell culture supports 116 and 116′ because of the thin thickness, which may lead to the detachment of cultured cells or dead space generated by contact between the cell culture supports. In addition, when the thickness is more than 800 μm, the number of cell culture supports stacked within a limited volume may be reduced, resulting in a decrease in the total effective culture area within the cell culture unit 110 with a limited volume. In addition, even when a fiber web is included in the cell culture supports, there is a concern that medium exchange may not occur smoothly in the thickness direction of the cell culture supports, which may reduce cell proliferation and EV production.

In addition, on the surfaces of the cell culture supports 116 and 116′, physiologically active ingredients that promote cell attachment, migration, proliferation, and EV production and secretion may be included.

The physiologically active ingredient may be any known component that promotes the attachment, migration and proliferation of stem cells, and EV production and secretion without limitation, and may be any one or more compounds selected from the group consisting of a monoamine, an amino acid, a peptide, a saccharide, a lipid, a protein, a glycoprotein, a glucolipid, a proteoglycan, a mucopolysaccharide, and a nucleic acid, which have these functions.

In addition, the physiologically active ingredient may be, specifically, a material that is present in the extracellular matrix or an artificially prepared material identical or similar thereto.

In addition, the physiologically active ingredient may include a physiologically active peptide, and the physiologically active peptide may include a motif. The motif may be a natural peptide or recombinant peptide including a predetermined amino acid sequence included in any one or more selected from growth factors, and/or proteins, glycoproteins and proteoglycans included in the extracellular matrix.

Specifically, the motif may include a predetermined amino acid sequence included in any one or more growth factors (GFs) selected from the group consisting of adrenomedullin, angiopoietin, a bone morphogenic protein (BMP), a brain-derived neurotrophic factor (BDNF), an epidermal growth factor (EGF), erythropoietin, fibroblast growth factor, a glial cell line-derived neurotrophic factor (GDNF), a granulocyte colony-stimulating factor (G-CSF), a granulocyte macrophage colony-stimulating factor (GM-CSF), growth differentiation factor-9 (GDF9), a hepatocyte growth factor (HGF), a hepatoma-derived growth factor (HDGF), an insulin-like growth factor (IGF), a keratinocyte growth factor (KGF), a migration-stimulating factor (MSF), myostatin (GDF-8), a nerve growth factor (NGF), a platelet-derived growth factor (PDGF), thrombopoietin (TPO), a T-cell growth factor (TCGF), neuropilin, transforming growth factor-α (TGF-α), transforming growth factor-β (TGF-β), tumor necrosis factor-α (TNF-α), a vascular endothelial growth factor (VEGF), IL-1, IL-2, IL-3, IL-4, IL-5, IL-6, and IL-7. In addition, the motif may include a predetermined amino acid sequence included in any one or more extracellular matrixes selected from the group consisting of hyaluronic acid, heparin sulfate, chondroitin sulfate, tertian sulfate, keratan sulfate, alginate, fibrin, fibrinogen, collagen, elastin, fibronectin, vitronectin, cadherin, and laminin.

In addition, the motif may include both a predetermined amino acid sequence included in a growth factor and a predetermined amino acid sequence included in the extracellular matrix.

Preferably, the physiologically active ingredient may be a fused oligopeptide or fused polypeptide in which an attachment peptide moiety derived from a mussel adhesive protein and a functional peptide moiety capable of promoting cell culture and production and secretion of EVs are fused, and specifically includes any one amino acid sequence selected from the group consisting of SEQ ID NOS: 1 to 3, or any one or more selected therefrom. Here, fusion may specifically involve the binding of a functional peptide moiety to the carboxyl terminus, the amino terminus, or both the carboxyl and amino termini of the attachment peptide. Here, the binding may be a covalent bond, and specifically, an amino bond. In addition, the functional peptide and the attachment peptide may be bonded by a known method, and for example, may be prepared by a recombinant protein production method using E. coli. In addition, the attachment peptide and the functional peptide may be linked by a direct covalent bond, but the present invention is not limited thereto, and may be linked by a linker. For example, the linker is a peptide linker, which may not have any specific biological activity other than linking the regions or conserving some minimal distance or other spatial relationship between them. However, the constituent amino acids of the linker may be selected to influence some properties of the molecule, for example, folding, net charge, or hydrophobicity. In addition, it is disclosed that the attachment peptide and the functional peptide can be indirectly linked via a predetermined material such as a crosslinking agent.

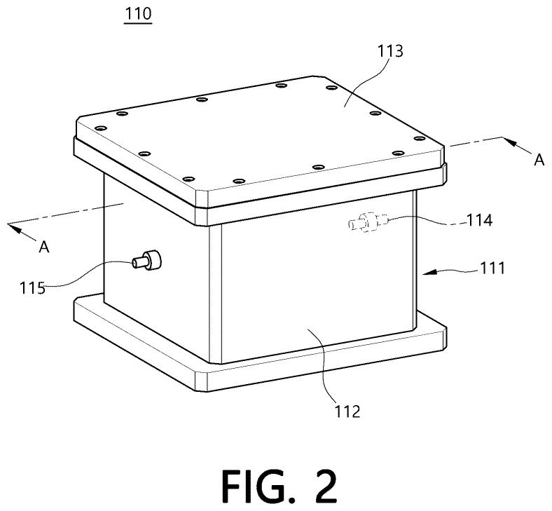

Referring to FIGS. 2 to 5, the cell culture unit 110 may include a culture housing 111 and the above-described the cell culture supports 116 and 116′, and the culture housing 111 may include an inlet 114 and an outlet 115 through which a medium 140 is introduced into an internal accommodation space S1 and discharged to the outside, i.e., a medium storage unit 120.

Specifically, the culture housing 111 may accommodate a certain amount of the medium 140 and a plurality of cell culture supports 116 and 116′ therein as described above. To this end, the culture housing 111 may be formed as a case with an accommodation space S1.

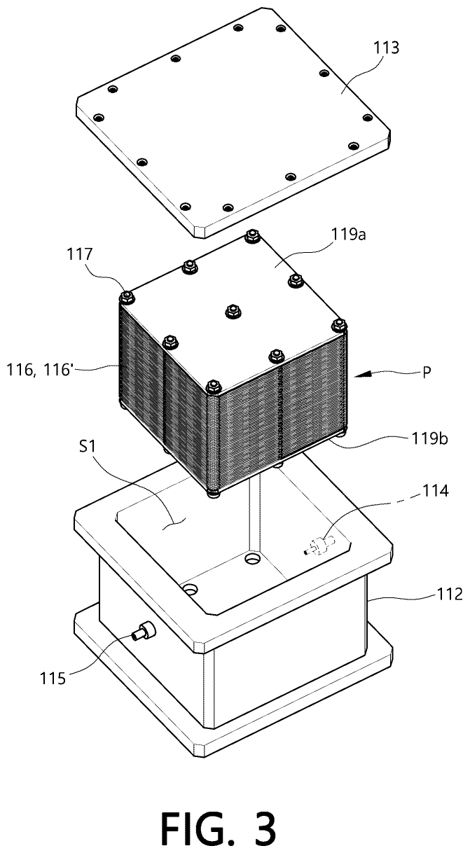

In one example, the culture housing 111 may include, as shown in FIG. 3, a housing body 112 in the form of a case with an accommodation space S1 with an open top. The inlet 114 and the outlet 115 may be formed at respective sides of the housing body 112, and the top-open accommodation space S1 may be sealed with a cover member 113 connected to the housing body 112.

Therefore, the medium 140 supplied from the medium storage unit 120 to the cell culture unit 110 may fill the accommodation space S1 through the inlet 114, and the medium 140 filled in the accommodation space S1 may be discharged to the outside through the outlet 115.

Here, the inside of the housing body 112 where the inlet 114 is formed may be formed to be recessed inward with the inlet 114 as a center. Specifically, the inside of the housing body 112 may be formed to have a cone or quadrangular pyramid shape so that the cross-sectional area gradually increases along the direction of medium movement from the end of the inlet 114, and the end of the inlet 114 may form the center of the cone or quadrangular pyramid shape.

In other word, the inside of the housing body 112 where the inlet 114 is formed may be formed concavely with the inlet 114 as a center in a direction opposite to the inflow direction of the medium. Therefore, the medium 140 introduced through the inlet 114 from the medium storage unit 120 may be smoothly introduced into the accommodation space S1.

Here, between the inlet 114 and the cell culture supports 116 and 116′ disposed in the accommodation space S1, a dispersion plate may be further disposed to disperse the medium introduced through the inlet 114. The dispersion plate may be disposed a certain distance from ends of the cell culture supports 116 and 116′ disposed in the accommodation space S1 of the housing body 112.

The dispersion plate may prevent the medium introduced from the outside through the inlet 114 from moving directly into the inside of the accommodation space S1, thereby evenly dispersing the medium introduced from the medium storage unit 120 through the inlet 114. Therefore, the evenly dispersed medium is allowed to move simultaneously to each cell culture support regardless of the location of the cell culture support disposed in the accommodation space S1 while passing through the dispersion plate, thereby smoothly supplying the above medium to each cell culture support, so that cells with a uniform size and properties may be cultured on a large scale regardless of the location of the cell culture supports where the cells are cultured.

In one example, the dispersion plate may include a plurality of through holes passing through the plate body with a predetermined area, but the present invention is not limited thereto. The dispersion plate may be a plate-shaped mesh net with a plurality of through holes.

Meanwhile, although it has been described that the inside of the housing body 112 has a cone or square pyramid shape, the present invention is not limited thereto. It is disclosed that the housing body 112 may be implemented so that a cap portion (not shown) with an internal cone- or square pyramid-shape protrudes outside the housing body 112.

In addition, in the cell culture unit 110, for mass production of cells and EVs secreted therefrom, the above-described plurality of plate-shaped cell culture supports 116 and 116′ may be arranged spaced apart in one direction in the accommodation space S1 in the culture housing 111. Specifically, the plurality of cell culture supports 116 and 116′ may be configured in an assembly form to increase integration and improve handling and assembly properties.

In one example, the cell culture support assembly P may be in an integrated form in which the plurality of cell culture supports 116 and 116′ are spaced apart from each other at predetermined intervals.

Particularly, the cell culture support assembly P may be configured in a laminate form in which the plurality of supports 116 and 116′ are disposed in parallel and spaced apart along the height direction of the housing body 112.

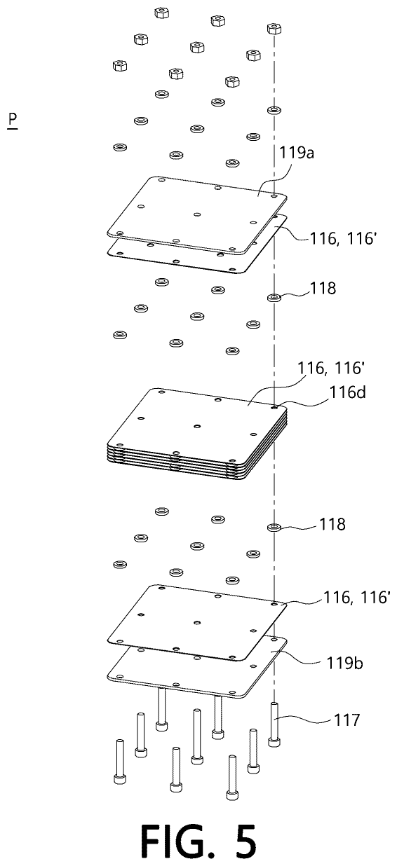

To this end, as shown in FIG. 5, the cell culture support assembly P may include a plurality of fastening bars 117 with a predetermined length and a plurality of spacing members 118 formed in a ring shape, and the fastening bars 117 may be inserted into the plurality of supports 116 and 116′.

In this case, the plurality of fastening bars 117 may be spaced apart from each other at predetermined intervals, and both ends of the fastening bars 117 may be fixed to an upper plate 119A and a lower plate 119B, which have a predetermined area, respectively.

Accordingly, the plurality of fastening bars 117 whose both ends are respectively fixed to the upper plate 119A and the lower plate 119B may remain spaced apart from each other, and the plurality of cell culture supports 116 and 116′ may be disposed in parallel between the upper plate 119A and the lower plate 119B. The plurality of supports 116 and 116′ may be fastened to the fastening bars 117 through a plurality of through holes 116D disposed through the supports at positions corresponding to respective fastening bars 117.

Moreover, the plurality of spacing members 118 may be fitted onto the plurality of fastening bars 117, respectively, like the cell culture supports 116 and 116′, and the plurality of spacing members 118 and the plurality of cell culture supports 116 and 116′ may be alternately fastened to each fastening bar 117.

Accordingly, the spacing members 118 may be disposed between two cell culture supports 116 and 116′ arranged parallel to each other, and the two cell culture supports 116 and 116′ disposed above and below may remain spaced apart by the spacing members 118.

The above-described cell culture support assembly P is not limited thereto, and any known method can be applied as long as the plurality of cell culture supports 116 and 116′ are arranged in parallel along one direction and remain spaced apart from each other at a certain interval.

Meanwhile, in the cell culture support assembly P, a distance between adjacent cell culture supports 116 and 116′ may be 5 mm or less, and more preferably, 0.8 to 3 mm, which may be advantageous to ensure that the cultured cells are appropriately stimulated by the medium flowing through the space between the adjacent cell culture supports 116 and 116′. Therefore, it may be advantageous for further promoting the production and secretion of target EVs such as exosomes. When the distance between the adjacent cell culture supports 116 and 116′ is more than 5 mm, there is a concern that the promotion of EV production and secretion is minimal, the number of cell culture supports 116 and 116′ which can be disposed in the accommodation space S1 in the limited cell culture unit may be reduced, thereby reducing the effective culture area, and when the effective culture area is set the same and the distance is increased, there is a risk of excessive medium consumption, which is not economical. Meanwhile, when the distance between the cell culture supports 116 and 116′ is less than 0.8 mm, the flow of medium may be even impeded, leading to increased back pressure, and there is a concern that contact between cell culture supports due to a medium passing through the narrow space between the cell culture supports and a dead space resulting therefrom may occur.

In addition, the medium storage unit 120 may include a medium 140 containing nutrients required for cell culture. The medium storage unit 120 may be connected to the above-described cell culture unit 110 through a connecting tube to provide the stored medium 140 to the cell culture unit 110. The medium storage unit 120 may include a medium housing 121 formed in a case shape with a storage space for storing a certain amount of the medium.

In this case, the medium housing 121 may include a medium inlet 123 and a medium outlet 122 through which the medium 140 may be introduced or discharged so that the medium 140 stored in the storage space may be recovered after being supplied into the cell culture unit 110.

Here, the medium inlet 123 may be connected to the outlet 115 of the cell culture unit 110, and the medium outlet 122 may be connected with the inlet 114 of the cell culture unit 110 via a circulation pump 130. Therefore, the medium 140 stored in the storage space of the medium housing 121 may return to the medium storage unit 120 after being introduced into the cell culture unit 110 through the operation of the circulation pump 130.

Meanwhile, the medium circulation-type EV production unit may further include a gas supply unit (not shown) that supplies a gas to the medium 140 to maintain a gas, e.g., carbon dioxide, in the medium 140 at a constant concentration. The gas supply unit may be connected to one side of the medium storage unit 120, and the gas supplied from the gas supply unit may be dissolved in the medium 140 stored in the medium storage unit 120 to maintain the concentration and pH of carbon dioxide dissolved in the medium 140 at a state suitable for cell culture. Particularly, the medium recovered from the cell culture unit 110 may have a diluted carbon dioxide concentration, but the medium returning to the medium storage unit 120 is advantageous in maintaining an appropriate carbon dioxide concentration and pH required for cell culture, for example, pH 7.5 to 8.0 and a CO₂ concentration of 4.5 to 6%, which are conditions suitable for performing Step (1) of the present invention, due to the gas supplied through the gas supply unit. Here, the gas supplied to the medium may be carbon dioxide, air, or a mixed gas in which various gases are mixed in a particular ratio depending on the purpose. In one example, the gas may consist of carbon dioxide. In addition, in one example, the mixed gas may be a gas in which carbon dioxide and oxygen are mixed in a predetermined ratio. Here, the oxygen concentration may be 2 to 14%, which may be more advantageous in achieving the purpose of the present invention.

Meanwhile, the medium 140 supplied to the cell culture unit 110 may not include bubbles. As described above, when the gas is supplied to the medium storage unit 120 through the gas supply unit, bubbles may be included in the medium 140, and the bubbles introduced into the cell culture unit 110 may interfere with cell culture or make uniform cell culture difficult.

To prevent bubbles from being included in the medium, the medium inlet 123 of the medium storage unit 120 may be formed in the medium housing 121 at a relatively higher position than the medium outlet 122. That is, the medium outlet 122 may be formed in the medium housing 121 relatively closer to the bottom surface of the medium housing 121 than the medium inlet 123. The medium inlet 123 may be formed at a position relatively farther from the bottom surface of the medium housing 121 than the medium outlet 122. Therefore, even when bubbles are introduced into the medium storage unit 120 due to a gas that is generated in a process of the medium circulating along a connection tube or a process of recovering the medium into the storage space through the medium inlet 123, and/or is supplied from the gas supply unit, the bubbles included in the medium may move upward due to buoyancy during the process of moving the medium to the medium outlet 122 formed at a relatively lower position than the medium inlet 123. Therefore, the medium discharged through the medium outlet 122, that is, the medium supplied to the cell culture unit 110, may not include bubbles.

As such, the medium circulation-type EV production unit according to one embodiment of the present invention may be implemented as a closed circulation system that is configured to circulate the medium 140 stored in the medium storage unit 120 by the circulation pump 130 after passing through the cell culture unit 110 to completely separate cells located in the cell culture unit 110 from the external environment.

Meanwhile, Step (1) may be performed by including a cell seeding step of supplying cells mixed in a medium to the cell culture unit 110, a cell culture step of producing EVs by circulating the medium 140 such that the medium 140 discharged through one side of the cell culture unit 110, that is, the outlet 115, is supplied again to the cell culture unit 110, and a step of obtaining a culture containing the produced EVs.

First, the cell seeding step may be performed by seeding cells mixed in a medium in the cell culture unit 110. In addition, after inputting the cells mixed in the medium to fill the cell culture unit 110, the supply of medium may be stopped for a certain time, for example, 30 minutes to 6 hours to allow the cells introduced into the cell culture unit 110 to attach to the surface of the cell culture supports. Here, the time for stopping the supply of medium may be appropriately changed in consideration of the amount of introduced cells, the volume of the accommodation space in the cell culture unit 110, the area and number of cell culture supports, and thus the present invention is not particularly limited thereto.

More specifically, in the cell seeding step, the medium containing cells may be provided while the main surface of each of the cell culture supports 116 and 116′ is erect with respect to the ground, for example, the cell culture unit 110 is oriented such that the main surface is substantially perpendicular to the ground. Therefore, this may be advantageous for cells to migrate uniformly between the cell culture supports of the cell culture support assembly P in which the plurality of cell culture supports are assembled at narrow intervals. In addition, in order to more stably attach the cells supplied and accommodated in the cell culture unit 110 on the cell culture supports 116 and 116′, while the supply of medium is stopped, the cell culture unit 110 may be left for a predetermined period of time such that the main surface of each of the cell culture supports 116 and 116′ lays down with respect to the ground, that is, substantially horizontal. Here, in the positional relationship between the main surface of each of the cell culture supports 116 and 116′ and the ground, the term “substantially perpendicular” or “substantially horizontal” means that the angle formed by the ground and the main surface is generally 90° or 0° as viewed by one of ordinary skill in the art. In one example, “substantially perpendicular” may mean that the angle between the two surfaces is more than 85° to 90°, and “substantially horizontal” may mean that the angle between the two surfaces is 0 to less than 5°.

In addition, as described above, the cell culture unit 110 may further include a rotation means for converting the direction of the main surface of each of the cell culture supports 116 and 116′ in the cell culture unit 110, and thus the cell seeding step may be more easily performed.

Here, the supplied cells may encompass immunocytes, embryonic stem cells, adult stem cells, induced pluripotent step cells (iPS cells), and progenitor cells, and for example, the stem cells may include one or more types selected from the group consisting of embryonic stem cells, adult stem cells, iPS cells, and progenitor cells. The stem cells may be homologous stem cells and/or autologous stem cells.

The immunocytes also called immune system cells, and may include immune cells known to be responsible for immunity, including NK cells, T cells, B cells, dendritic cells, and macrophages.

The embryonic stem cells are stem cells derived from fertilized eggs, and are stem cells with the ability to differentiate into cells of all types of tissue.

In addition, the induced pluripotent stem cells (iPS cells) are called reverse differentiation stem cells, and refer to cells that are induced to have pluripotency like embryonic stem cells by injecting differentiation-related genes into differentiated somatic cells and returning them to the cell stage before differentiation.

In addition, the progenitor cells have the ability to differentiate into a specific type of cells like stem cells, but they are more specific and targeted compared to stem cells, and unlike stem cells, they have a finite number of divisions. The progenitor cells may be mesoderm-derived progenitor cells, but are not limited thereto. In the specification, the progenitor cells be included in the category of stem cells, and unless otherwise specified, “stem cells” are interpreted as a concept that also includes progenitor cells.

In addition, adult stem cells are stem cells extracted from umbilical cord blood or adult bone marrow, blood, nerves, etc., and refer to primitive cells just before differentiation into cells of a specific organ. The adult stem cells may be one or more types selected from the group consisting of hematopoietic stem cells, mesenchymal stem cells, and neural stem cells. The adult stem cells may be mammalian adult stem cells, for example, human adult stem cells. Adult stem cells are difficult to proliferate and have a strong tendency to differentiate easily. However, using various types of adult stem cells, not only can they regenerate various organs required in actual medicine but they can also differentiate according to the characteristics of each organ after transplantation, and thus can be advantageously applied to the treatment of incurable diseases.

In one example, the adult stem cells may be mesenchymal stem cells, for example, human mesenchymal stem cells. Mesenchymal stem cells (MSCs) are called mesenchymal stromal cells (MSCs), and refer to multipotent stromal cells that can differentiate into various types of cells such as osteoblasts, chondrocytes, myocytes, and adipocytes. MSCs may be pluripotent cells derived from non-marrow tissues such as the placenta, umbilical cord blood, adipose tissue, adult muscle,
corneal stroma, and the dental pulp of baby teeth.

Subsequently, in step 1-2), a cell culture step of producing EVs by circulating the medium 140 discharged through the outlet 115 of the cell culture unit 110 so that it is supplied again to the cell culture unit 110 is performed. Specifically, the medium discharged through the outlet 115 of the cell culture unit 110 may be recovered in the medium storage unit 120, adjusted to have appropriate levels of carbon dioxide concentration and pH for cell culture and EV production/secretion, and then supplied again to the inlet 114 of the cell culture unit 110.

In one example, step 1-2) may be performed for 4 to 6 days, but the present invention is not limited thereto.

In addition, step 1-2) may be performed at 37 to 38° C., and the flow rate of the circulating medium may be 20 to 150 mL/min, and preferably, 20 to 100 mL/min, which is advantageous for culturing cells capable of producing and secreting EVs with a target size. Meanwhile, a specific flow rate of the medium in the above flow rate range may be determined by comprehensively considering the volume of the accommodation space S1 of the cell culture unit 110, the number and spacing of the cell culture supports 116 and 116′ disposed in the accommodation space S1, and the amount of the medium filling the rest of the accommodation space S1 while accommodating the cell culture support assembly P. For example, as the number of sections of the cell culture support assembly P increases, the amount of medium accommodated increases, and the spacing between the cell culture supports increases, the medium flow rate may be adjusted to be faster.

In addition, in step 1-2), after proliferating cells to occupy 80% to 90% the total effective cell culture area, for example, the effective cell culture area of cell culture supports when the plurality of cell culture supports are disposed in the cell culture unit 110, the circulating medium may be controlled not to contain foreign EVs. This means that secretion of EVs can begin or increase after cell proliferation has occurred to a certain extent, and when the medium contains EVs derived from foreign cells or xenobiotics, rather than the cells cultured in the medium, it is difficult to completely obtain EVs derived from the cultured cells. Particularly, in the early stage of cell culture, medium components required for cell culture, such as fetal bovine serum, may be inevitably included in the medium, and EVs contained in fetal bovine serum and EVs secreted from the cultured cells are mixed and may be difficult to distinguish. After cell proliferation for a certain period of time, cells may be cultured in a medium not containing foreign EVs, for example, cultured while foreign EVs are removed from the medium by filtration or a serum-free medium is exchanged and circulated. Meanwhile, when cells are cultured while the medium is circulated with a medium containing foreign EVs and then exchanged with a medium not containing foreign EVs, the cells may be further cultured for 20 to 50 hours after medium exchange, but the present invention is not limited thereto.

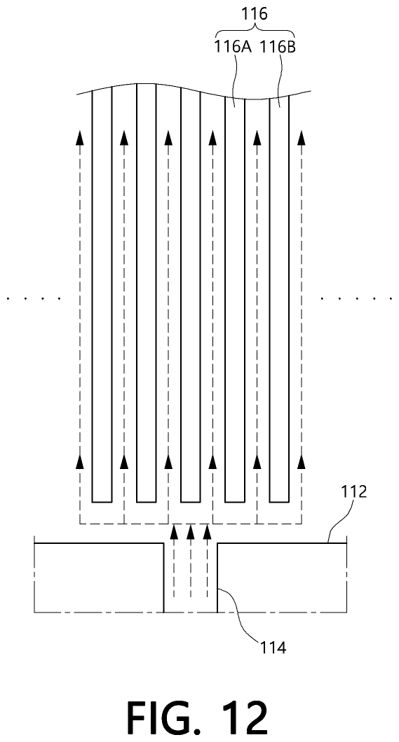

In addition, referring to FIG. 12, in step 1-2), the cell culture unit 110 may be oriented such that the main surface of each of the plurality of plate-shaped cell culture supports 116 accommodated therein is substantially perpendicular to the ground. This is advantageous for culturing cells with high EV production because the medium is supplied to the inlet 114 located on the lower side of the cell culture unit 110 which has been oriented as described above, flows upward through the space between the adjacent cell culture supports 116A and 116B and then discharged through the outlet located on the upper side of the cell culture unit 100. In other words, the flow caused by the circulating medium is favorable for increasing the amount of EVs with a target size, which are produced and secreted from cells by stimulating the culture cells with shear force. In addition, when the medium flowing into the cell culture unit 110 is discharged through the space between the cell culture supports, EV production is promoted in each cultured cell regardless of the location of the plurality of cell culture supports spaced apart from each other because uniform medium flow and the generation of uniform stimuli applied to the cells are possible regardless of the location of the space between the cell culture supports, so that there are advantages of increasing the total amount of EVs obtained, and increasing the amount of target EVs. In step 1-2), when the medium is circulated while the cell culture unit 110 is oriented such that the main surface of the cell culture supports is tilted close to the ground, the increase in EV production may be insignificant. In addition, when the cell culture unit 110 is oriented such that the main surface of the cell culture supports is substantially perpendicular to the ground, and the inlet 114 through which the medium is introduced and the outlet 115 are formed on the lower and upper sides of the cell culture unit 110, the medium can be circulated to flow through the space between the cell culture supports in a bottom-to-top direction from the ground. Therefore, even when gas bubbles are included in the medium, the bubbles introduced into the cell culture unit may rapidly move toward the outlet 115, which is the opposite direction to gravity, and may be easily discharged to the outside, so that the gas bubbles do not escape the cell culture unit and remain inside, which is advantageous for minimizing concerns about cell culture inhibition.

Next, as step 1-3), the step of obtaining a culture containing produced EVs is performed. The culture may be the medium accommodated in the cell culture unit 110 and the medium storage unit 120 after completing the cell culture, and to obtain it, the medium in the cell culture unit 110 may be recovered into the medium storage unit 120. In addition, the produced EVs may be cell-derived constructs in the form of a particle in which a variety of biomolecules, including proteins with various functions (e.g., various types of growth factors, chemokines, cytokines, and transcription factors), which are released (secreted) from the cultured cells to the extracellular environment, RNAs (mRNA and miRNA), and lipids, are encapsulated by a cell membrane with the same lipid bilayer as that of the cell from which it was derived, but the present invention is not limited thereto, and they may include all things commonly referred to as EVs regardless of their origin, secretion mechanism, and size.

Next, in Step (2) according to the present invention, a step of separating and obtaining EVs with a size of 200 nm or less, including exosomes, from a stock solution containing one or more of the culture obtained in Step (1) and a cell lysate derived from the cells is performed.

As described above, in the culture finally obtained in Step (1) or a cell lysate derived from the cultured cells, EVs generally called EVs regardless of their size are included, and other than these, non-lysed cells, a cell lysate, and a non-target component such as a protein may also be included. Therefore, in Step (2), a process of separating and obtaining target EVs, for example, EVs with a size of 200 nm or less, including exosomes, preferably, EVs with a size of 50 to 200 nm, from a stock solution including any one or more of the culture containing EVs obtained in Step (1) and a cell lysate derived from the cultured cells may be performed.

The stock solution includes a culture and/or a cell lysate derived from the cultured cells. The cell lysate may be obtained by a known cell lysis method after obtaining the cells cultured in Step (1), but the present invention is not particularly limited thereto. However, to increase the yield of EVs with a target size of 200 nm or less, a cell lysis unit where cells are lysed by applying a physical force to the cells may be used, and specifically, cells may be lysed using cavitation, shear force, or impact.

In one example, the cell lysis may be performed according to a lysing method by injecting a cell solution including cultured cells into various channels with a larger width than the size of the cultured cell under high pressure. The cell lysis performed by such a method may use, as a specific example, a high-pressure spray.

Alternatively, in another example, the cell lysis may be performed according to a lysing method by forcibly passing cells through a channel with a smaller width than the cell size. The cell lysis performed by such a method may use, for a specific example, a known cell extruder.

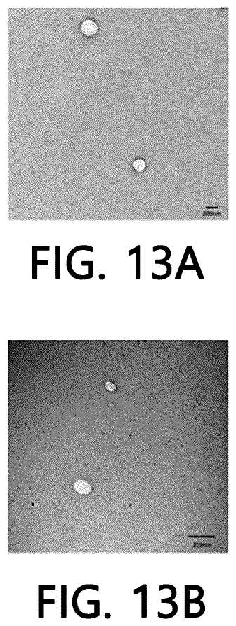

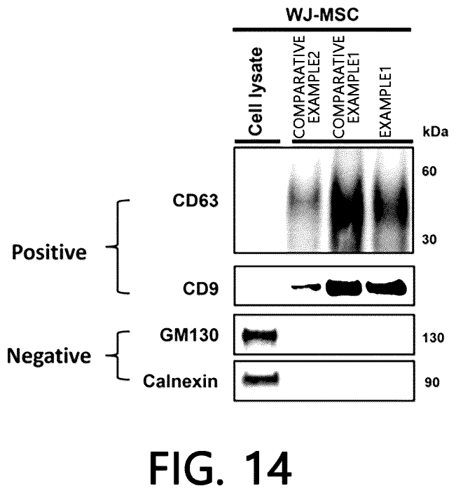

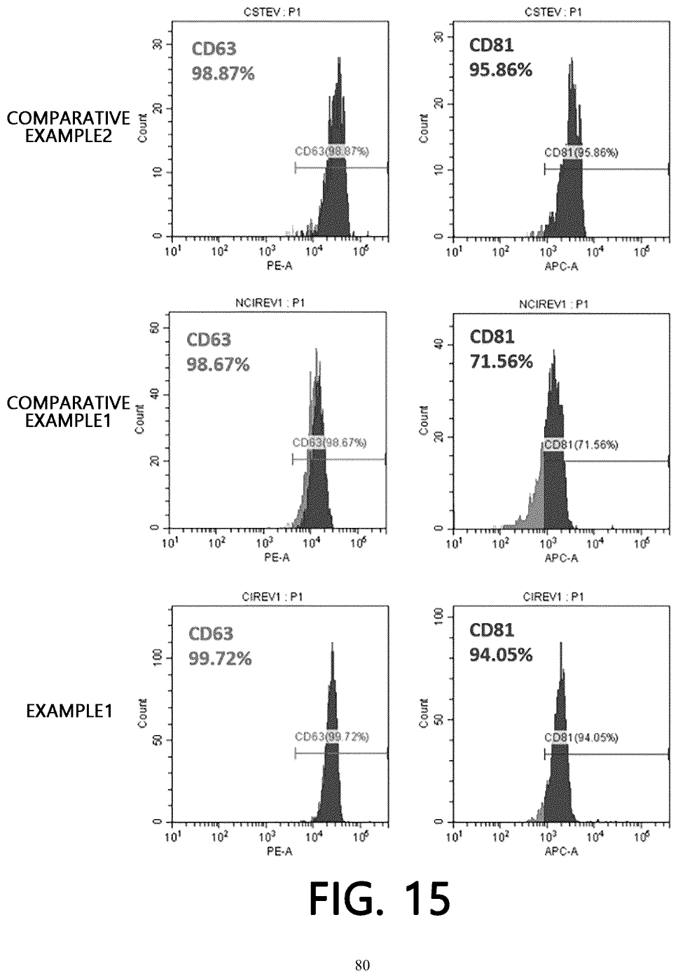

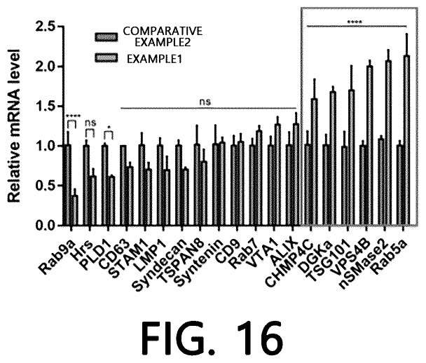

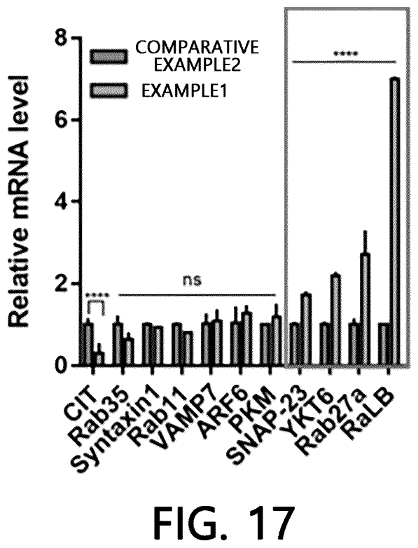

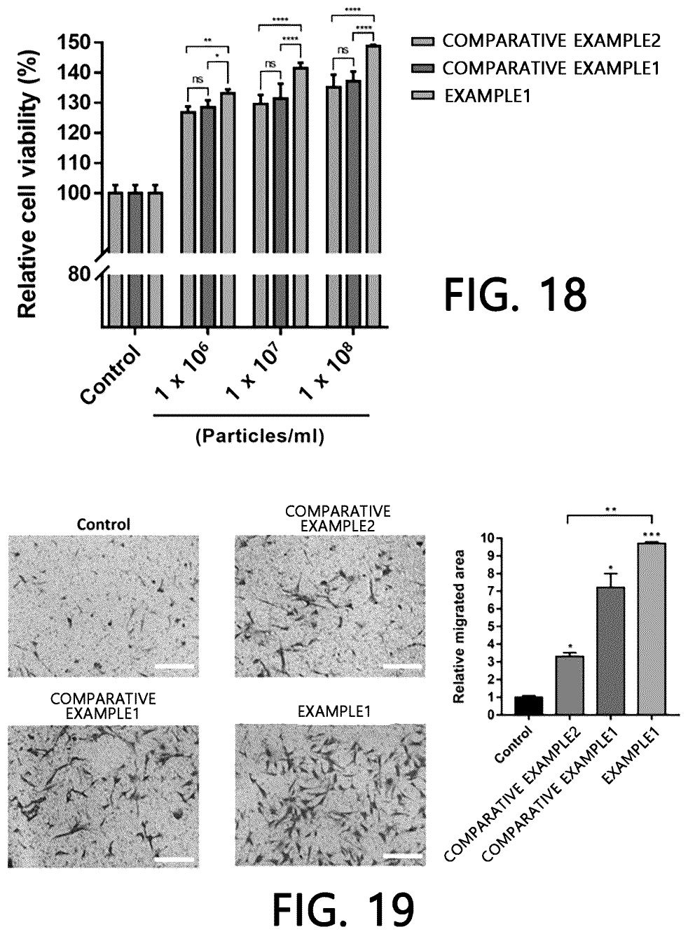

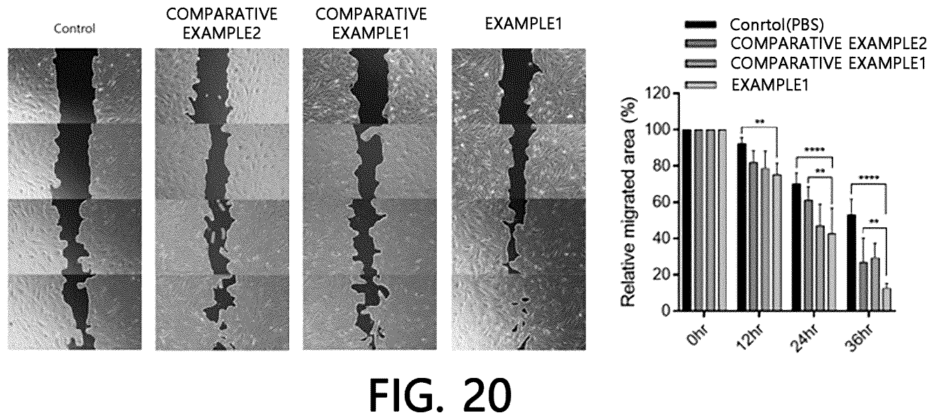

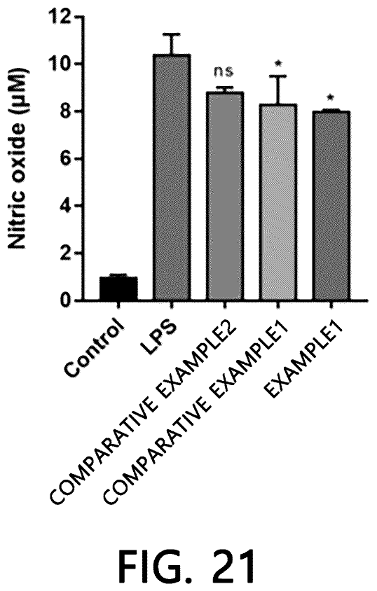

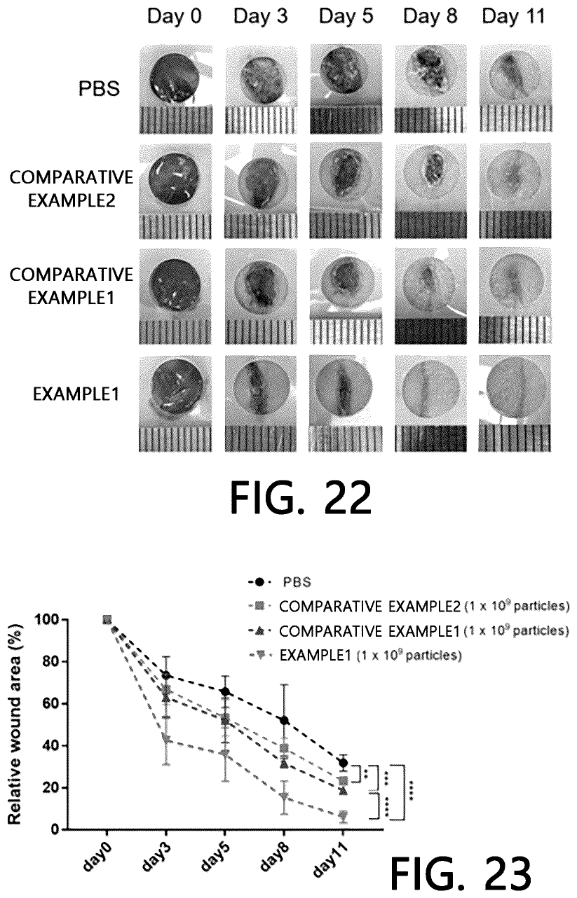

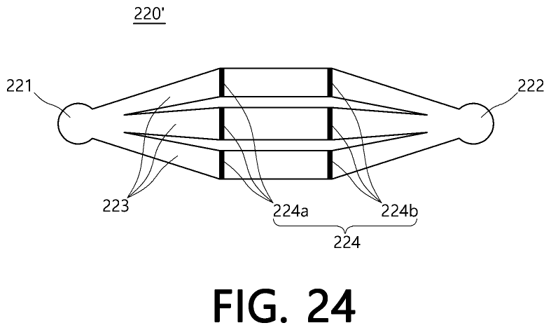

Meanwhile, in the method of lysing cells by forcibly passing through the channel with a smaller width than the cell size, cell lysis using a known cell extruder may not have a high yield of EVs with a desired size of 200 nm or less. According to one embodiment of the present invention, a cell lysis unit 220′ with a microchannel as shown in FIG. 13 may be used, and thus is advantageous for improving the yield of EVs with a size of 200 nm or less by allowing cells to pass through the cell lysis unit 220′ several times.

Specifically, cell lysis unit 220′ may include an inlet 221 through which a cell solution, which is a lysis target, is introduced, an outlet 222 through which a cell lysate is discharged, a flow channel 223 through which the lysis target passes, which connects the inlet 221 and the outlet 222, and has a width of 1 mm or less, and a partition 224 which is disposed to block the flow channel 223 and perforated with a plurality of microchannels for lysing cells in the lysis target. Here, when several partitions 224 are arranged in a row in one flow channel 223 to increase the cell lysis efficiency, there is a risk of the flow channel collapsing, and therefore, there are three flow channels 223 linking the inlet 221 and the outlet 222, and a plurality of partitions 224 may be included in each flow channel 223.

In addition, to improve the yield of EVs with a size of 200 nm or less by repeating cell lysis in which the cell lysate passing through the cell lysis unit 220′ passes through the cell lysis unit 220′ again, the plurality of partitions 224 disposed in one flow channel 223 include a first partition 224A and a second partition 224B disposed closer to the outlet 222 than the first partition 224A, wherein the width of a microchannel formed in the first partition 224A and the width of a microchannel formed in the second partition 224B may be different from each other. More preferably, the width of a microchannel formed in the first partition 224A may be 10 to 20 μm, and the width of a microchannel formed in the second partition 224B may be 1 to 5 μm. Through this, it can be advantageous for greatly increasing the yield of EVs having a target size of 200 nm or less from the cell lysate obtained by repeated lysis. When the widths of the microchannels in the plurality of partitions are all the same, the yield of EVs with a target size of 200 nm or less may be almost unchanged even when repeated lysis is performed.

The stock solution containing the cell lysate obtained by the above-described method and/or the culture obtained in Step (1) may undergo a first filtration step for separating a first filtrate containing first particles with a size of 200 nm or less, including EVs, and a second filtration step for separating and removing second particles with a size of less than 50 nm from the first filtrate, and additionally, a third filtration step for separating and removing third particles with a size exceeding 200 nm from the second filtrate that has undergone the second filtration step, thereby finally obtaining EVs with a size of 50 to 200 nm.

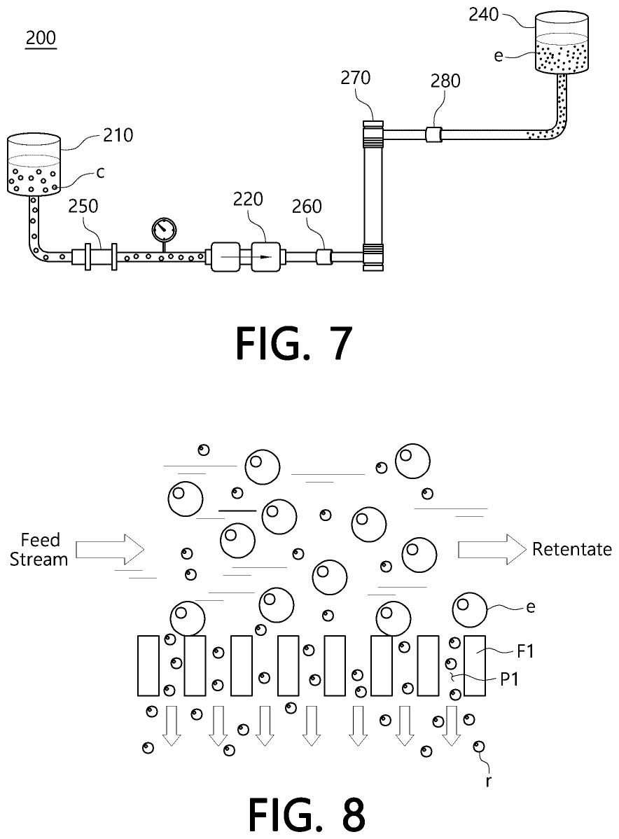

The first filtration step, the second filtration step, and the additional third filtration step may be performed using separation units 200, 201, and 202 as shown in FIGS. 7 to 11.

Specifically, the separation unit 200, 201, or 202 may include a first filter unit 260 or 260′ in which the first filtration step is performed to separate and obtain first particles with a size of 200 nm or less, a second filter unit 270 or 270′ in which the second filtration step is performed to separate and remove second particles with a size of less than 50 nm from the first filtrate containing the separated first particles, and a recovery unit 240 in which the separated EVs with a size of 50 to 200 nm are stored.

In addition, the first filter unit 260 or 260′ may be connected to a raw material supply unit 210 for supplying the stock solution to the first filter unit 260 or 260′. When the filtration target is a culture, the culture corresponding to the stock solution may be stored in the raw material supply unit 210. Alternatively, when the filtration target includes a cell lysate derived from the cultured cells, a cell lysate corresponding to the stock solution may be included, or the cultured cells or a culture containing the cultured cells may be stored in the raw material supply unit 210, and a cell lysis unit 220 may be further disposed between the raw material supply unit 210 and the first filter unit 260 or 260′ to supply the cell lysate to the first filter unit 260 or 260′. Here, when the cultured cells are stored in the raw material supply unit 210, the cells to be lysed may be stored while being mixed in a medium or buffer.

In addition, a pump 250 for supplying the raw materials stored in the raw material supply unit 210 to the cell lysis unit 220 or the first filter unit 260 or 260′ may be further included between the raw material supply unit 210 and the first filter unit 260 or 260′.

The first filter unit 260 or 260′ is a filtering device for separating and obtaining first particles with a size of 200 nm or less from the stock solution, and for example, as shown in FIG. 7, the first filter unit 260 may include a plate-shaped filtering member having a plurality of pores with a predetermined size to filter EVs with a size exceeding 200 nm included in the stock solution or other cell lysate, and may consist of a known syringe filter, bottle filter, and flat membrane filter, including such a filtering member.

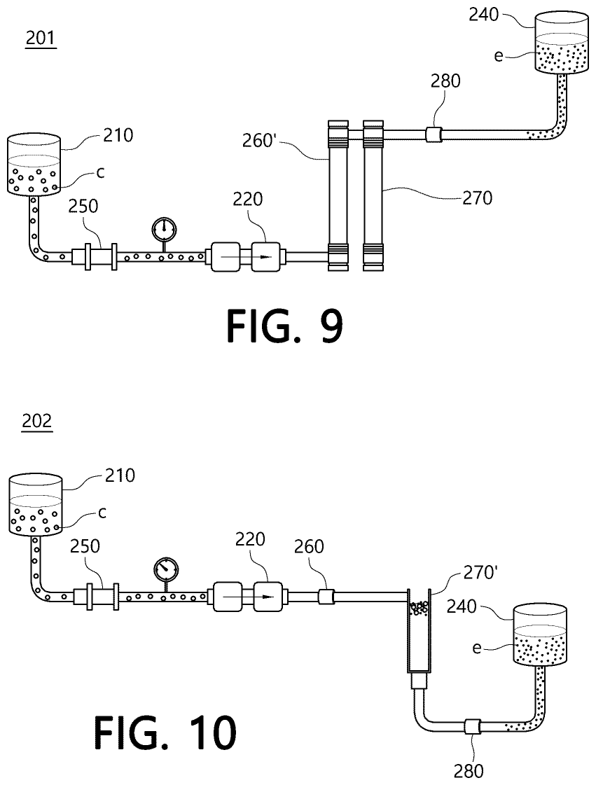

In addition, the first filter unit 260′ may include a tangential flow filtration-type filtering device as shown in FIG. 9 to obtain a first filtrate containing first particles with a size of 200 nm or less in the stock solution. The tangential flow filtration method may use a known TFF filtering device to further filter the first particles by filtering smaller materials than the pores of the filter included in the filtering device in a direction perpendicular to the direction in which the stock solution, which is the solution to be filtered, flows, as shown in FIG. 8, for example, the first particles, concentrating particles larger than the first particles in a solution to be filtered, and purifying the concentrated solution to be filtered through recirculation. In addition, other than the first filter unit, the tangential flow filtration method may be performed by a known filtration method such as size exclusion chromatography as described below, and the present invention is not particularly limited thereto.

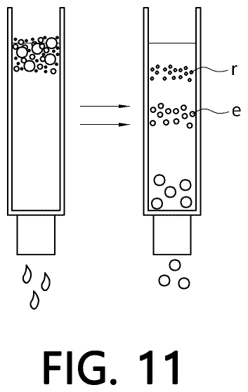

In addition, the second filter unit may serve to separate and remove second particles with a size of less than 50 nm from the first filtrate containing the separated first particles, and the second particles with a size of less than 50 nm may be proteins, lipids, and cell fragments. In one example, as shown in FIG. 9, the second filter unit 270 may serve to filter second particles (r), which are smaller materials than the pores of the filter included in the filtering device in a direction perpendicular to the direction in which the first filtrate, which is a solution to be filtered, flows, by employing the tangential flow filtration method shown in FIG. 8, and concentrate particles (e) larger than the second particles (r) in the first filtrate to separate and remove particles with a smaller size than the desired target size. In addition, as shown in FIG. 10, the second filter unit 270′ may employ the size exclusion chromatography method shown in FIG. 11. Such types of filtration are more advantageous in that desired particles can be rapidly separated and protected compared to the conventional filtration method using a separation membrane in which the flow of the stock solution and the filtration direction of the particles are the same in order to separate and obtain the filtrate containing the particles (e) with a size of 50 to 200 nm larger than the second particles smaller than the target size, for example, with a size of less than 50 nm, in the first filtrate using the second filter unit. That is, according to the conventional filtration method using a separation membrane in which the flow of the stock solution and the filtration direction of the particles are the same, the particles with a target size of 50 to 200 nm remain on the separation membrane and the particles with a size of less than 50 nm are obtained in a filtrate, such that the target particles are likely damaged or their yield is reduced by separating and obtaining the remaining target particles on the separation membrane again, and the filtration time is likely extended due to re-separation.

Meanwhile, when a TFF filtering device, which is a tangential flow filtration type, is employed as a second filter unit 270, a plate-type (cassette-type) filter may be included, and in this case, it may be more advantageous to separate and obtain EVs of a target size compared to a hollow filter.

In addition, when size exclusion chromatography is employed as a second filter unit 270′, known size exclusion chromatography using a porous resin may be employed. The porous resin may include pores with a certain size, and the pores may be formed to have a relatively larger size than second particles with a size of less than 50 nm to be separated and removed and have a relatively smaller size than a target size. Therefore, in the process in which the first filtrate passes through the second filter unit 270′, the second particles with a size of less than 50 nm may first be removed from the first filtrate, and a second filtrate from which the second particles are removed may be separated and obtained. That is, during the process in which the second particles with the relatively smallest size in the first filtrate are subjected to size exclusion chromatography, the second particles may move relatively more slowly than the first particles which have a relatively larger size, and therefore, a mixture containing particles with a size of 50 to 200 nm may be discharged first in size exclusion chromatography.

Next, at the rear end of the second filter unit 270 or 270′, a third filter unit 280 in which a third filtration step for separating and removing third particles with a size exceeding 200 nm in the second filtrate that has undergone the second filtration step may be performed may be further included. The third filter unit 280 may employ a conventional filtration method for separating and removing particles larger than a desired size, and for example, a filtering device employing the above-described filtration method in the first filter unit may be used, and the present invention is not particularly limited thereto.

In the filtrate passing through the above-described second filter unit 270 or 270′ or the third filter unit 280, EVs with a size of 50 to 200 nm, including exosomes, are included, and may be separated and stored in the recovery unit 240.

Meanwhile, as described above, from the stock solution that includes one or more of the culture containing EVs obtained in Step (a) and a cell lysate derived from the cultured cells, EVs with a target size are separated and obtained using a separation unit 200, 201, or 202, which at least includes the above-described first filter unit 260 or 260′ and the second filter unit 270 or 270′. When the concentration of the particles in the stock solution is high, even by filtering through the same first filter unit 260 or 260′ and second filter unit 270 or 270′, there is a concern that the yield of the finally obtained EVs with a size of 200 nm or less may be significantly reduced. This is because aggregation between particles in the stock solution may occur during the filtration process, and when EVs with a target size are included in the aggregated particles, the content of EVs with a target size in the final product is bound to be reduced, and when the concentration of the stock solution is high, such a phenomenon may probably worsen. In addition, when the concentration of particles filtered from the stock solution is high, the filtered particles may be attached to a filtering member to form a cake. In this case, EVs with a target size may be captured in the cake without passing through the filtering member, and thus the content of EVs with a target size in the final product may be reduced.

Therefore, according to one embodiment of the present invention, in the first filtration step, to appropriately control the amount of particles with a size exceeding 200 nm, which remain on the filtering member without passing through it, multi-step filtration may be performed by configuring a first filter unit by arranging a plurality of filtering members to remove smaller particles for each step, so that particles with a size more than and close to 200 nm are ultimately excluded. Through this, the capture of particles to be filtered on the cake formed on the filtering member may be minimized.

In addition, the first filtration step may be performed with a stock solution diluted 3.0 to 15.0 times with a buffer, and thus, the occurrence of aggregation between particles contained in the stock solution may be minimized, and even when a plurality of filtering members are not arranged, the yield of EVs with a target size may be improved by minimizing the cake formed on the filtering member. When the stock solution is diluted less than 3.0 times, the improvement in yield of EVs having a final desired size of 200 nm or less may be insignificant. In addition, when the stock solution is diluted more than 15 times, the improvement in yield of EVs may be insignificant.

In addition, according to another embodiment of the present invention, the first filtration step may be performed while a stock solution is not diluted, and in the second filtration step, a first filtrate introduced to a second filter unit 270 or 270′ may be previously diluted, and specifically, the first filtrate diluted 3.0 to 15.0 times with a buffer may be introduced into the second filter unit 270 or 270′, thereby improving the yield of the final EVs with a target size. When the first filtrate is diluted less than 3.0 times, the improvement in yield of EVs with a finally desired size of 200 nm or less may be insignificant. In addition, when the first filtrate is diluted more than 15 times, the improvement in EV yield may be insignificant.

However, preferably, compared to performing the second filtration step with the diluted first filtrate, performing the first filtration step with the diluted stock solution is more advantageous in improving the yield of finally obtained EVs with a target size. Particularly, when cells are mass-cultured in Step (1), the concentrations of various types of particles contained in the stock solution including the culture obtained in Step (1) and a cell lysate of the cultured cells may be high, and in these cases, it may be more advantageous for obtaining a high yield of EVs with a target size.

Meanwhile, in this case, when the second filtration step or the third filtration step is further performed, a dilution method for diluting a filtrate obtained before storage in a recovery unit after the third filtration step with a large amount of buffer may be further included, and in this process, the medium contained in the filtrate may be exchanged and removed with a buffer.

EVs with a size of 200 nm or less, and particularly, EVs obtained by culturing stem cells, obtained by the method described above, may be included as an active ingredient of a composition for antiinflammation or regenerating damaged tissue, and thus the composition may be used as a composition for preventing or treating inflammation, or regenerating damaged tissue such as a wound.

The composition may be administered to a subject via various administration routes such as oral administration or parenteral administration, and for example, may be injected or implanted into a lesion of the subject, or administered via a parenteral administration route such as intravascular administration (intravenous or intraarterial administration) or subcutaneous administration, but the present invention is not limited thereto. In addition, the subject to be administered the composition may be an animal selected from mammals such as primates including a human and monkeys, and rodents including rats and mice, or birds and/or tissue or cells derived (isolated) from the animal, or their cultures. In addition, EVs, for example, stem cell-derived EVs, included as an active ingredient in the composition may be stem cells of the same species as the subject, autologous stem cells, or a mixture thereof.

In addition, EVs with a size of 200 nm or less obtained by the above-described method may be contained in a medium composition for cell culture as an additive for cell culture that can improve cell proliferation to express a cell culture enhancing effect close to existing fetal bovine serum (FBS), and thus can be useful as an additive for a cell culture medium that can replace FBS.

Table 1 below shows amino acid sequences of the above-described physiologically active peptides.

TABLE 1

| SEQ | |
|---|---|
| ID | |
| NO: | Amino acid sequence |
| 1 | Met Ala Ser Ala Lys Pro Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr Lys Gly Cys Ser |
| | Ser Glu Glu Tyr Lys Gly Gly Tyr Tyr Pro Gly Asn Ala Tyr His Tyr His Ser Gly Gly Ser Tyr |
| | His Gly Ser Gly Tyr His Gly Gly Tyr Lys Gly Lys Tyr Tyr Gly Lys Ala Lys Lys Tyr Tyr Tyr |
| | Lys Tyr Lys Asn Ser Gly Lys Tyr Lys Tyr Leu Lys Lys Ala Arg Lys Tyr His Arg Lys Gly |
| | Tyr Lys Lys Tyr Tyr Gly Gly Ser Ser Glu Phe Glu Phe Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr Lys Ala Lys Pro |
| | Ser Tyr Pro Pro Thr Tyr Lys Lys Leu Gly Arg Gly Asp Ser Pro |
| 2 | Met Ala Ser Ala Lys Pro Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr Lys Gly Cys Ser |
| | Ser Glu Glu Tyr Lys Gly Gly Tyr Tyr Pro Gly Asn Ala Tyr His Tyr His Ser Gly Gly Ser Tyr |
| | His Gly Ser Gly Tyr His Gly Gly Tyr Lys Gly Lys Tyr Tyr Gly Lys Ala Lys Lys Tyr Tyr Tyr |
| | Lys Tyr Lys Asn Ser Gly Lys Tyr Lys Tyr Leu Lys Lys Ala Arg Lys Tyr His Arg Lys Gly |
| | Tyr Lys Lys Tyr Tyr Gly Gly Ser Ser Glu Phe Glu Phe Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr Lys Ala Lys Pro |
| | Ser Tyr Pro Pro Thr Tyr Lys Lys Leu Pro His Ser Arg Asn |
| 3 | Met Ala Ser Ala Lys Pro Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr Lys Gly Cys Ser |
| | Ser Glu Glu Tyr Lys Gly Gly Tyr Tyr Pro Gly Asn Ala Tyr His Tyr His Ser Gly Gly Ser Tyr |
| | His Gly Ser Gly Tyr His Gly Gly Tyr Lys Gly Lys Tyr Tyr Gly Lys Ala Lys Lys Tyr Tyr Tyr |
| | Lys Tyr Lys Asn Ser Gly Lys Tyr Lys Tyr Leu Lys Lys Ala Arg Lys Tyr His Arg Lys Gly |
| | Tyr Lys Lys Tyr Tyr Gly Gly Ser Ser Glu Phe Glu Phe Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr Lys Ala Lys Pro |
| | Ser Tyr Pro Pro Thr Tyr Lys Lys Leu Gly Arg Gly Asp Ser Pro Pro His Ser Arg Asn |

### EXAMPLES

The present invention will be described in further detail with reference to the following examples, but the examples are not intended to limit the scope of the present invention, and should be interpreted as helping understand the present invention.

### Example 1

As shown in FIG. 1, a bioreactor (FIG. 13, AMOLIFESCIENCE, HPB-11-15) corresponding to a medium circulation-type EV production unit including a cell culture unit (accommodation space volume: 200 cm³), a medium storage unit and a circulation pump was prepared. Subsequently, as a cell culture support disposed in the cell culture unit, a fiber web formed of PVDF fibers, which have an average diameter of 260 nm, a basis weight of 4.5 g/m² and a thickness of 5 μm, was laminated with a polycarbonate (PC) film with a thickness of 450 μm with a silicone-based adhesive on one side, and then attached and integrated using a coating device (Digital-3500Plus) at room temperature, thereby manufacturing a laminate-type cell culture support, and then the support was punched to have a size of 11 cm×11 cm (width× length). After the punched cell culture support fiber web was coated with a physiologically active ingredient having the amino acid sequence represented by SEQ ID NO: 3 in Table 1, as shown in FIG. 5, 15 cell culture supports were spaced 1 mm apart from each other and stacked in a plurality in a culture housing, and then the culture housing was sealed from the outside air. Afterward, the cell culture unit was oriented so that the inlet provided on one side of the culture housing faced the ground and the main surface of the cell culture support was perpendicular to the ground, the medium mixed with mesenchymal stem cells (MSCs) derived from umbilical cord blood was input through the inlet to fill the accommodation space of the culture housing, and the cell culture unit was oriented so that the main surface of the cell culture support in the cell culture unit was horizontal to the ground. After being left for 4 hours, the cell culture unit was reoriented such that the main surface of the cell culture support was perpendicular to the ground and the inlet faced the ground, and the cells were cultured at 37° C. for 4 hours while re-circulating a pure medium without MSCs using a circulation pump at 20 mL/min.

Here, a carbon dioxide concentration and pH were adjusted by supplying carbon dioxide gas from a gas supply unit to a medium storage unit to adjust the carbon dioxide concentration and pH in the medium to 5% and 7.8, respectively. In addition, the medium was 10% FBS (16000044, Gibco)-containing DMEM LOW (D6046, Sigma), and MSCs to be seeded were mixed in the medium to be 4,000 cells/cm² per unit area of the cell culture support.

After culturing to 90% of the total effective culture area of the cell culture supports included in the cell culture unit, the medium was exchanged with FBS-free DMEM LOW (D6046a, Sigma) and then recirculated for 24 hours to prepare a culture containing EVs, and then the medium contained in the cell culture unit was returned to the medium storage unit, thereby obtaining a culture containing EVs stored in the medium storage unit.

In addition, a 0.15% Trypsin-EDTA solution whose temperature was adjusted to 37° C. was injected into the cell culture unit and left for 15 minutes to separate the cultured MSCs from the cell culture support and suspend them, FBS-free DMEM LOW (D6046, Sigma) was injected to neutralize the injected trypsin component, and the medium containing the cultured MSCs was harvested.

Subsequently, a first filtrate from which particles with a size exceeding 200 nm were removed by filtering 360 mL of the culture containing EVs through a first filter unit, which is a 0.2 μm bottle top filter (431153, Corning), was provided to a second filter unit (Pall, Minimate™ TFF capsules) using a tangential flow filtration method and employing a plate-type (cassette-type) TFF filter, thereby obtaining 120 mL of a 3× concentrated second filtrate from which second particles with a size of less than 50 nm were removed. 720 mL of a PBS buffer was injected into the second filtrate to dilute the medium and concentrated 6 times again using the second filter unit, thereby obtaining a second filtrate in which 120 mL of the medium was exchanged, and then the same filter unit employed in the first filter unit was disposed in the third filter unit to additionally remove particles with a size exceeding 200 nm, which may remain in the second filtrate in which the medium was exchanged, thereby separating and obtaining EVs with a size of 50 to 200 nm.

### Example 2

EVs were prepared in the same manner as in Example 1, except that a filtrate containing EVs with a final size of 50 to 200 nm was separated and obtained by circulating a medium while maintaining the orientation of the cell culture unit so that the main surface of a cell culture support was horizontal to the ground, instead of orienting the cell culture unit so that the main surface of the cell culture support in the cell culture unit was perpendicular to the ground before medium circulation begins after seeding MSCs and leaving them for 4 hours.

### Comparative Example 1

EVs were prepared in the same manner as in Example 1, but after injecting a medium mixed with MSCs into the cell culture unit, MSCs were proliferated to 90% of the total effective culture area of the cell culture support at 37° C. without circulating the medium, and then the medium was exchanged in the same manner as in Example 1, but the exchanged medium was not circulated but allowed to stand for 24 hours, and then the medium in the cell culture unit was separated to obtain an EV-containing culture and a medium containing cultured stem cells. Among these, from the culture, a filtrate containing EVs with a final size of 50 to 200 nm was separated and obtained in the same manner as in Example 1.

### Comparative Example 2

EVs were prepared in the same manner as in Example 1, except that the incubator was changed to CellSTACK (3269, Corning), a non-medium-circulation-type cell incubator equipped with a support that was not coated with a physiologically active ingredient, MSCs were cultured to 90% of the effective culture area. The medium was then exchanged in the same manner as in Example 1 and allowed to stand for 24 hours. Afterward, the medium was separated to obtain a culture containing EVs, and from the culture, a filtrate containing EVs with a final size of 50 to 200 nm was separated and obtained in the same manner as in Example 1.

### <Experimental Example 1>

Filtrates containing EVs obtained according to Examples 1 and 2 and Comparative Examples 1 and 2 were subjected to characteristic analysis.

1. Number analysis of EVs

30 mL of the filtrate in each filtration step in each Example and Comparative Example was sampled to measure the number of particles included in the sample using Zeta View (Particle Metrix, Germany), and the number of separated particles containing EVs at each filtration step is shown in Table 2 below.

TABLE 2

| Number of particles | | | Comparative | Comparative |
|---|---|---|---|---|
| per step | Example 1 | Example 2 | Example 1 | Example 2 |
| Particles in filtrate | 2.53 × 10¹² | 2.35 × 10¹² | 1.99 × 10¹¹ | 2.16 × 10¹¹ |
| after passing through | (11.67) | (10.89) | (0.92) | (1) |
| first filter unit | | | | |
| (200 nm or less)(number/mL) | | | | |
| (relative multiple) | | | | |
| Particles in filtrate | 1.38 × 10¹² | 1.06 × 10¹² | 1.69 × 10¹¹ | 1.38 × 10¹¹ |
| after passing through | (10.04) | (7.68) | (1.23) | (1) |
| second filter unit | | | | |
| (50 nm or more)(number/mL) | | | | |
| (relative multiple) | | | | |
| Particles in filtrate | 3.75 × 10¹¹ | 3.04 × 10¹¹ | 8.42 × 10¹⁰ | 8.01 × 10¹⁰ |
| after passing through | (4.68) | (3.80) | (1.05) | (1) |
| third filter unit | | | | |
| (50~200 nm)(number/mL) | | | | |
| (relative multiple) | | | | |
| Average size of particles | 91.0 | 95.0 | 100.1 | 103.7 |
| in filtrate after passing | | | | |
| through third filter | | | | |
| unit (nm) | | | | |

As confirmed from Table 2, it can be seen that the number of EVs with a size of 50 to 200 nm in Example 1 where cells were cultured by circulating a medium and produced and secreted EVs is 4.45 times larger than that of Comparative Example 1 where the same cells were cultured using the same device without medium circulation and produced/secreted EVs, and 4.68 times larger than that of Comparative Example 2 where the same cells were cultured and produced/secreted EVs using a different type of incubator without medium circulation.

Meanwhile, from the results shown in Table 2, when a first filtration step using a first filter unit and a second filtration step using a second filter unit were performed, it can be confirmed that there are a considerable amount of particles with a size exceeding 200 nm in the obtained second filtrate, implying that the aggregation of the particles included in the filtrate may occur during each filtration step, and it can be seen that the yield of actually obtained EVs with a size of 50 to 200 nm, which are not aggregated and separated, may be reduced due to aggregation.

### Example 3

A filtrate containing EVs with a final size of 50 to 200 nm was prepared in the same manner as in Example 1, except that the filtrate was separated and obtained after changing the number of cell culture supports accommodated in the cell culture unit (accommodation space volume: 200 cm³) to 12 (effective culture area: 1452 cm²), and setting a medium flow rate to 20 mL/min.

### Example 4

A filtrate containing EVs with a final size of 50 to 200 nm was prepared in the same manner as in Example 3, except that the filtrate was separated and obtained after changing the cell culture support accommodated in the cell culture unit to a polycarbonate film with the same thickness, which was coated with the same physiologically active ingredient.

### Example 5

A filtrate containing EVs with a final size of 50 to 200 nm was prepared in the same manner as in Example 3, except that the filtrate was separated and obtained after changing the cell culture support accommodated in the cell culture unit to a plasma-treated polycarbonate film, which was not coated with a physiologically active ingredient.

### <Experimental Example 3>

For Examples 3 to 5, the number and diameter of particles contained in the filtrate were analyzed in the same manner as in Experimental Example 1. The results are shown in Table 4 below.

TABLE 4

| Number of particles | | | |
|---|---|---|---|
| per step | Example 3 | Example 4 | Example 5 |
| Particles in filtrate | 1.99 × 10¹² | 1.60 × 10¹² | 1.53 × 10¹² |
| after passing through | (130.1) | (104.58) | (100) |
| first filter unit | | | |
| (200 nm or less)(number/mL) | | | |
| (relative percentage(%)) | | | |
| Particles in filtrate | 1.29 × 10¹² | 1.06 × 10¹² | 7.50 × 10¹¹ |
| after passing through | (172.0) | (141.3) | (100) |
| second filter unit | | | |
| (50~200 nm)(number/mL) | | | |
| (relative percentage(%)) | | | |
| Particles in filtrate | 5.57 × 10¹¹ | 2.79 × 10¹¹ | 2.77 × 10¹¹ |
| after passing through | (201.1) | (100.1) | (100) |
| third filter unit | | | |
| (50~200 nm)(number/mL) | | | |
| (relative multiple) | | | |

As can be seen in Table 4, when a cell culture support including a physiologically active ingredient-coated fiber web having nanofibers with an average diameter of 260 nm was employed as in Example 1, compared to Example 4 that employed a cell culture support, which is a PC film treated with plasma only, the target EVs were obtained at a level increased to 201.0%, compared to the EV particles with a size of 50 to 200 nm.

In addition, in Example 3 employing the physiologically active ingredient-coated PC film as a cell culture support, a similar amount of target EVs was obtained compared to Example 4, but it can be seen that the yield is significantly lower than in Example 1.

### Example 6

A filtrate containing EVs with a final size of 50 to 200 nm was prepared in the same manner as in Example 3, except that the filtrate was separated and obtained after diluting the first filtrate three-fold by mixing with PBS in an amount twice the volume of the first filtrate and passing it through the second filter unit instead of providing it directly to the second filter unit.

### Example 7

A filtrate containing EVs with a final size of 50 to 200 nm was prepared in the same manner as in Example 6, except that the filtrate was separated and obtained after changing a dilution ratio as shown in Table 5 below.

### Example 8

A filtrate containing EVs with a final size of 50 to 200 nm was prepared in the same manner as in Example 3, except that the filtrate was separated and obtained after increasing the number of cell culture supports accommodated in the cell culture unit (accommodation space volume: 700 cm³) to 47, changing a medium flow rate during medium circulation to 40 mL/min, and passing the first filtrate diluted three-fold by mixing with PBS in an amount twice the volume of the first filtrate as described in Example 6 through the second filter unit.

### Example 9

A filtrate containing EVs with a final size of 50 to 200 nm was prepared in the same manner as in Example 8, except that the filtrate was separated and obtained after changing the type of seeded cells to umbilical cord-derived MSCs.

### <Experimental Example 4>

The number and size of particles in the filtrates obtained in each filtration step in Examples 3, and 6 to 9 were analyzed in the same manner as in Experimental Example 1. The results are shown in Table 5 below.

TABLE 5

| Number of particles | | | | | |
|---|---|---|---|---|---|
| per step | Example 3 | Example 6 | Example 7 | Example 8 | Example 9 |
| Number of supports | 12 | 12 | 12 | 47 | 47 |
| for cell culture | | | | | |
| Whether to dilute | Not | Diluted | Diluted | Diluted | Diluted |
| first filtrate | diluted | three-fold | two-fold | three-fold | three-fold |
| Particles in first | 1.99 × 10¹² | 1.99 × 10¹² | 1.99 × 10¹² | 2.96 × 10¹² | 3.43 × 10¹² |
| filtrate after | | | | | |
| passing through | | | | | |
| first filter unit | | | | | |
| (200 nm or less)(number/mL) | | | | | |
| Particles in second | 1.29 × 10¹² | 1.69 × 10¹² | 1.38 × 10¹² | 2.46 × 10¹² | 2.99 × 10¹² |
| filtrate after | (35.2%) | (15.1%) | (30.7%) | (16.9%) | (12.8%) |
| passing through | | | | | |
| second filter unit | | | | | |
| (50~200 nm) (number/mL) | | | | | |
| (Reduction rate compared | | | | | |
| to number of particles | | | | | |
| in first filtrate) | | | | | |
| Particles in third | 5.57 × 10¹¹ | 9.87 × 10¹¹ | 6.29 × 10¹¹ | 2.05 × 10¹² | 2.58 × 10¹² |
| filtrate after | | | | | |
| passing through | | | | | |
| third filter unit | | | | | |
| (50~200 nm)(number/mL) | | | | | |

As can be seen in Table 5, in Examples 6, 8, and 9 in which the first filtrate passing through the first filter unit was diluted three-fold before being input to the second filter unit and then passed through the second filter unit, the number of particles was greatly reduced compared to Example 3. In addition, ultimately, it can be seen that the number of EVs with a size of 50 to 200 nm, which were finally obtained through the third filter unit, in Example 6 was increased 77.1% compared to Example 3, which was obtained under the same conditions.

However, it can be seen that the number of particles in Example 7 where the dilution ratio was set to 2-fold was significantly reduced compared to that in the first filtrate in Example 6, and the number of finally obtained EVs with a size of 50 to 200 nm was also decreased.

### Example 10

A filtrate containing EVs with a final size of 50 to 200 nm was prepared in the same manner as in Example 9, except that the filtrate was separated and obtained after providing a stock solution diluted three-fold by mixing the culture containing EVs as a stock solution, with PBS, instead diluting the first filtrate, to the first filter unit.

### Examples 11 to 13

A filtrate containing EVs with a final size of 50 to 200 nm was prepared in the same manner as in Example 10, except that the filtrate was separated and obtained after changing the dilution ratio of a stock solution as shown in Table 6 below.

### <Experimental Example 4>

The number and size of particles in the filtrates obtained in each filtration step in Examples 9 to 13 were analyzed in the same manner as in Experimental Example 1. The results are shown in Table 6 below.

TABLE 6

| Number of particles | | | | | |
|---|---|---|---|---|---|
| per step | Example 9 | Example 10 | Example 11 | Example 12 | Example 13 |
| Number of supports | 47 | 47 | 47 | 47 | 47 |
| for cell culture | | | | | |
| Whether to dilute | Not | Diluted | Diluted | Diluted | Not |
| stock solution | diluted | three-fold | two-fold | six-fold | diluted |
| Whether to dilute | Diluted | Not | Not | Not | Not |
| first filtrate | three-fold | diluted | diluted | diluted | diluted |
| Particles in filtrate | 2.58 × 10¹² | 4.1 × 10¹² | 3.0 × 10¹² | 4.2 × 10¹² | 1.04 × 10¹² |
| after passing through | | | | | |
| third filter unit | | | | | |
| (50~200 nm | | | | | |
| (number/mL) | | | | | |

As can be seen in Table 6, in Example 10 in which the first to third filtration steps are performed after diluting the stock solution, the amount of finally obtained EVs with a size of 50 to 200 nm was increased 58.9%, compared to Example 9 in which the second and third filtration steps are performed after diluting the first filtrate.

In addition, even when the stock solution was diluted, it can be seen that, in Example 11 in which two-fold dilution is performed, the amount of finally obtained EVs with a size of 50 to 200 nm was decreased compared to Example 10.

### Example 14

100 mL of a cell solution prepared by mixing the medium containing cultured MSCs, obtained in Example 1, with PBS to have the final concentration of 2.0×10⁵ cells/mL was prepared and injected into an inlet of a cell lysis unit as shown in FIG. 13, and the cell lysate obtained through an outlet was passed through the cell lysis unit an additional three times, thereby obtaining a final cell lysate. Here, the cell lysis unit included three flow channels in which an inlet diameter is 1 mm, a diameter of the outlet through which the cell lysate is discharged is 3 mm, and a width between the inlet and the outlet and through which a lysis target passes is 1 mm, a first partition which is disposed to block each flow channel and has a microchannel width of 10 μm, and a second partition which is disposed closer to the outlet than the first partition and has a microchannel width of 2 μm.

Afterward, a filtrate containing particles with a size of 200 nm or less, including EVs, was obtained by passing 10 mL of the extracted cell lysate as a stock solution through the first filter unit, which is a 250 mm syringe filter (PALL Acrodisc, the pore size of used filter: 0.2 μm).

### Example 15

A filtrate containing particles with a size of 200 nm or less, including EVs, was obtained in the same manner as in Example 14, except that the cell lysate was obtained in the same manner after changing the cell lysis unit to a cell lysis unit in which two partitions, which are first partitions, were disposed per flow channel without using a second partition, and passing it through the first filter unit in the same manner as described in Example 14.

### Example 16

A filtrate containing particles with a size of 200 nm or less, including EVs, was obtained in the same manner as in Example 14, except that the cell lysis unit was changed to a cell extruder (Avanti Polar Lipids, Extruder Set with Holder/Heating Block), a cell lysate was obtained by passing it through the cell extractor four times, and then passing it through a first filter unit in the same manner as described in Example 14.

### <Experimental Example 7>

The number of particles with a size of 200 nm or less included in the filtrates obtained in Examples 14 to 16 was measured using a particle analyzer (NanoSight NS300). The results are shown in Table 7 below.

In addition, to compare the number of particles with a size of 200 nm or less between the cell culture and cell lysate, the number of particles with a size of 200 nm, obtained by passing the culture obtained in Example 1 or Comparative Example 2 through the first filter unit was calculated based on the number of cells cultured at the recovery of the culture. The results are shown in Table 7 below.

TABLE 7

| | | | | | Comparative |
|---|---|---|---|---|---|
| | Example 15 | Example 16 | Example 17 | Example 1 | Example 2 |
| Whether to pass through | Pass | Pass | Pass | Pass | Pass |
| first filter unit | | | | | |
| Number of particles | 48,000/ | 41,500/ | 38,000/ | 20,000/ | 2,000/ |
| contained in sample | 200 nm or less | 200 nm or less | 200 nm or less | 200 nm or less | 200 nm or less |
| (number/cell)/size | | | | | |

As can be seen in Table 7, in Examples 15 to 17 in which particles were separated and obtained from a cell lysate derived from the cultured cells, the number of particles with a size of 200 nm or less per cultured cell is higher than the results of separating and obtaining particles from the culture in Example 1 and Comparative Example 2.

Meanwhile, among Examples 14 to 16, in Examples 14 and 15 in which the separation process was performed using a cell lysate obtained by lysis in a cell lysis unit having microchannels according to one embodiment of the present invention, it can be seen that higher lysis efficiency and a larger content of particles obtained with a size of 200 nm or less are achieved, compared to Example 16 in which the separation process was performed using a cell lysate lysed by a cell extruder.

### Example 17

A cell culture medium composition was prepared by mixing particles with a size of 200 nm or less obtained in Example 14 with DMEM to have a concentration of 1.0×10⁹ cells/mL in the total volume.

### Example 18

Particles with a size of 200 nm or less were prepared in the same manner as in Example 14, and mixed in DMEM to have a concentration of 0.5×10⁹ cells/mL in the total volume, thereby preparing a cell culture medium composition.

### Comparative Example 3

A cell culture medium composition was prepared by adding fetal bovine serum (FBS) to DMEM at a concentration of 1.0%.

### Comparative Example 4

A cell culture medium composition was prepared by adding FBS to DMEM at a concentration of 10.0%.

### <Experimental Example 8>

The effect of particles with a size of 200 nm or less, obtained in Examples 17 to 18 and Comparative Examples 3 to 4, on the cell culture of human dermal fibroblasts (HDFs) was investigated by comparing the proliferation amount after culturing HDFs under the same conditions, and the results are shown in Table 8 below.

TABLE 8

| | | | Comparative | Comparative |
|---|---|---|---|---|
| | Example 17 | Example 18 | Example 3 | Example 4 |
| Cell culture medium | Particles with | Particles with | FBS/1.0% | FBS/10.0% |
| additive (type/content) | size of 200 nm or | size of 200 nm or | | |
| | less/concentration | less/concentration | | |
| | 1.0 × 10⁹ | 0.5 × 10⁹ | | |
| | number/mL | number/mL | | |
| Relative proliferation | 128% | 116% | 100% | 154% |
| rate (%) of human dermal | | | | |
| fibroblast (HDF) | | | | |

As shown in Table 8, in the case of Examples 17 and 18 in which particles with a size of 200 nm or less, including EVs, were included as a medium additive, it can be confirmed that human dermal fibroblast (HDF) proliferation is improved, and compared to Comparative Example 4, it can be expected that the particles with a size of 200 nm or less can be used as a medium additive that can replace FBS when adjusting the effective particle concentration.

Although embodiments of the present invention have been described above, the spirit of the present invention is not limited thereto, and it will be understood by those of ordinary skill in the art that the present invention may be modified and altered in various ways by adding, altering, or deleting components without departing from the spirit of the present invention defined in the appended claims, and such modifications and alterations will also be included in the scope of the present invention.

What is claimed is:

1. An extracellular vesicle production system, comprising:
a medium circulation-type extracellular vesicle (EV) production unit for producing a culture containing EVs by culturing cells in a circulating medium; and
a separation unit for separating and obtaining the EVs with a size of 200 nm or less, comprising exosomes, from at least one stock solution selected from a recovered culture and a cell lysate derived from cultured cells.

2. The EV production system of claim 1, wherein the medium circulation-type EV production unit comprises a cell culture unit, a medium storage unit, a circulation pump provided, wherein a medium stored in the medium storage unit circulates between the cell culture unit and the medium storage unit, and a gas supply unit supplying a gas to the medium provided to the cell culture unit.

3. The EV production system of claim 2, wherein the cell culture unit comprises a culture hosing with a culture space and a plurality of plate-shaped cell culture supports spaced 5.0 mm or less apart in a plurality of stages inside the culture housing.

4. (canceled)

5. The EV production system of claim 1, wherein the separation unit comprises a first filter unit for separating and obtaining first particles with the size of 200 nm or less from the stock solution, a second filter unit for separating and removing second particles with a size of less than 50 nm from separated first particles, and a recovery unit for storing separated the EVs with a size of 50 to 200 nm.

6. The EV production system of claim 5, wherein the separation unit further comprises a third filter unit for separating third particles with a size exceeding 200 nm between the second filter unit and the recovery unit.

7. The EV production system of claim 5, wherein the second filter unit is configured to employ one or more of tangential flow filtration and size exclusion chromatography.

8. The EV production system of claim 5, wherein the separation unit further comprises a cell lysis unit for lysing the cultured cells by applying a physical force to the cultured cells or a culture containing the cultured cells, wherein the cultured cells and the culture containing the cultured cells are supplied from the medium circulation-type EV production unit, and the cell lysate produced by the cell lysis unit is supplied to the first filter unit.

9. The EV production system of claim 8, wherein the cell lysis unit comprises an inlet, an outlet, at least three fluid channels, and a plurality of partitions disposed to block each fluid channel, wherein a lysis target is supplied through the inlet, the cell lysate is discharged through the outlet, the lysis target passes through the at least three fluid channels, the at least three fluid channels connect the inlet and the outlet and have a width of 1 mm or less, the plurality of partitions have a plurality of microchannels passing through the plurality of partitions to lyse cells in the lysis target, and the plurality of partitions comprise a first partition and a second partition disposed closer to the outlet than the first partition, and a width of each microchannel formed in the first partition is 10 to 20 μm, and a width of each microchannel formed in the second partition is 1 to 5 μm.

10. A method of producing extracellular vesicles (EVs), comprising:
(1) producing a culture comprising the EVs by culturing cells in a circulating medium; and
(2) separating and obtaining the EVs with a size of 200 nm or less, comprising exosomes, from a stock solution comprising one or more selected from the culture and a cell lysate derived from cultured cells.

11. The method of claim 10, wherein the step (1) comprises a cell seeding step for supplying cells mixed in a medium to a cell culture unit, a cell culture step for producing the EVs by circulating the medium, wherein the medium discharged through ene a side of the cell culture unit is supplied back to the cell culture unit, and a step of obtaining a culture containing produced EVs.

12. The method of claim 10, wherein the medium is circulated at a rate of 20 to 100 mL/min, maintained at a pH of 7.5 to 8.0 with a CO₂ concentration of 4.5 to 6%, and the cells are cultured at 37 to 38° C.

13. The method of claim 11, wherein no bubbles are contained in the medium supplied to the cell culture unit.

14. The method of claim 10, wherein the cells are cultured on a plurality of plate-shaped cell culture supports, main surfaces of the plurality of plate-shaped cell culture supports face other at a predetermined interval and the main surfaces are arranged perpendicular to a ground, and the medium is circulated to flow through a space between the plurality of plate-shaped cell culture supports in a bottom-to-top direction from the ground.

15. The method of claim 11, wherein the cell culture unit comprises a plurality of plate-shaped cell culture supports, main surfaces of the plurality of plate-shaped cell culture supports face other at a predetermined interval,
the cell seeding step includes orienting the cell culture unit so that the main surfaces of the plurality of plate-shaped cell culture supports are substantially parallel to a ground to settle the cells onto the plurality of plate-shaped cell culture supports, and
the cell culture step includes culturing the cells by orienting the cell culture unit so that the main surfaces of the plurality of plate-shaped cell culture supports are substantially perpendicular to the ground.

16. The method of claim 11, wherein in the cell culture step, the cells are proliferated to occupy 80 to 90% of a total effective cell culture area in the cell culture unit, and then a medium not containing foreign EVs is circulated.

17. The method of claim 10, wherein the step (2) comprises a first filtration step for separating a first filtrate comprising first particles with the size of 200 nm or less, comprising the EVs, from the stock solution; and
a second filtration step for separating and removing second particles with a size of less than 50 nm from the first filtrate.

18. The method of claim 17, wherein the second filtration step is performed on the first filtrate diluted 3.0-fold to 15.0-fold with a buffer.

19. The method of claim 17, wherein the first filtration step is performed by multi-stage filtration of the stock solution to produce a filtrate containing smaller particles at each stage, or performed with the stock solution diluted 3.0-fold to 15.0-fold with a buffer.

20. The method of claim 17, further comprising:
a third filtration step for separating and removing third particles with a size exceeding 200 nm from a second filtrate undergone the second filtration step.

21. (canceled)

22. A medium composition for cell culture, comprising particles with a size of 200 nm or less, comprising extracellular vesicles, as a cell culture additive.


SEQUENCE LISTING (3 sequences)

SEQ ID NO: 1 - protein, 212 aa, synthetic construct
MASAKPSYPPTYKAKPSYPPTYKAKPSYPPTYKAKPSYPPTYKAKPSYPPTYKAKPSYPPTYKGCSSEEYKGGYYPGNAYHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKKLGRGDSP

SEQ ID NO: 2 - protein, 211 aa, synthetic construct
MASAKPSYPPTYKAKPSYPPTYKAKPSYPPTYKAKPSYPPTYKAKPSYPPTYKAKPSYPPTYKGCSSEEYKGGYYPGNAYHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKKLPHSRN

SEQ ID NO: 3 - protein, 217 aa, synthetic construct
MASAKPSYPPTYKAKPSYPPTYKAKPSYPPTYKAKPSYPPTYKAKPSYPPTYKAKPSYPPTYKGCSSEEYKGGYYPGNAYHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKKLGRGDSPPHSRN